(19) United States
(12) Patent Application Publication
(10) Pub. No.: US 2016/0237398 A1
(43) Pub. Date: Aug. 18, 2016

(54) METHODS OF MICROBIAL PRODUCTION OF EXCRETED PRODUCTS FROM METHANE AND RELATED BACTERIAL STRAINS

(71) Applicant: UNIVERSITY OF WASHINGTON THROUGH ITS CENTER FOR COMMERCIALIZATION, Seattle, WA (US)

(72) Inventors: Marina Kalyuzhnaya, Seattle, WA (US); Mary E. Lidstrom, Seattle, WA (US)

(73) Assignee: UNIVERSITY OF WASHINGTON THROUGH ITS CENTER FOR COMMERCIALIZATION, Seattle, WA (US)

(21) Appl. No.: 15/029,968

(22) PCT Filed: Oct. 20, 2014
(86) PCT No.: PCT/US14/61304

§ 371 Date: Apr. 15, 2016

Related U.S. Application Data
(60) Provisional application No. 61/892,909, filed on Oct. 18, 2013.

(51) Int. Cl. C12N 1/20; C12P 7/02; C12P 7/40

(52) U.S. Cl. CPC C12N 1/20; C12P 7/40; C12P 7/02

(57) ABSTRACT

The present disclosure is directed to methods of producing excreted products through the fermentation of methane with methanotrophs. In certain embodiments, the methods are performed at low oxygen levels.

### CROSS-REFERENCE TO RELATED APPLICATIONS

This application claims benefit under 35 U.S.C. §119(e) of U.S. Provisional Application No. 61/892,909 filed Oct. 18, 2013, the contents of which are incorporated herein by reference in their entirety.

### GOVERNMENT SUPPORT

This invention was made with federal funding under Grant Nos. MCB-0842686, awarded by the National Science Foundation, and under DE-SC0005154, awarded by the Department of Energy. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Oct. 16, 2014, is named 034186-082620-PCT_SL.txt and is 30,731 bytes in size.

### TECHNICAL FIELD

The technology described herein relates to the microbial conversion of methane to, e.g., organic acids and/or alcohols.

### BACKGROUND

Methane is an essential component of the global carbon cycle and one of the most powerful greenhouse gases, yet it is also one of the most promising alternative sources of carbon for biological production of chemicals of high added value. Aerobic methane-consuming bacteria (methanotrophs) represent a potential biological platform for methane-based biocatalysis.

Nature provides two alternative forms of methane as a resource: natural gas, relatively abundant today but still a nonrenewable fossil fuel, and renewable bio-gas, a byproduct of modern society that is often wasted [1-3]. Interest in new technologies for effective conversion of flared/wasted sources of methane into chemical compounds, including next-generation fuels, continues to increase [4-6]. The use of microbial cells and enzymes as catalysts for methane conversion represents an appealing approach in this context [7-11]. The benefits of methane biotechnology include a self-sustainable component, since any biomass generated could be used as single cell protein or converted back to methane via anaerobic digestion. However, besides single cell protein and polyhydroxybutyrate, exploitation of methane-based catalysis for the production of chemicals and fuels has not yet proven successful at the commercial level.

Gammaproteobacterial methanotrophs with the ribulose monophosphate (RuMP) pathway are among the most promising microbial systems for methane-based biotechnology. Reconstruction of the methane utilization network in these methanotrophs has been based on a number of biochemical studies that pointed toward the Entner-Doudoroff (EDD)-variant of the RuMP pathway as the major route for single carbon (C₁)-assimilation [12-14]. The major biochemical evidence that favored the EDD variant of the RuMP cycle included relatively high activities of two key enzymes of the pathway (6-phosphogluconate dehydratase and 2-keto-3-deoxy-6-phosphogluconate aldolase) and multiple enzymatic lesions in the Embden-Meyerhof-Parnas (EMP) pathway [15-16]. Activity of pyruvate kinase has not been detected previously in any gammaproteobacterial methanotrophs [16-17]. The presence of a reversible pyrophosphate (PPi)-dependent phosphofructotransferase led to the conclusion that the EMP pathway represents a metabolic loop balancing the level of glyceraldehyde-3-phosphate (GAP) and phosphoenolpyruvate (PEP) [16,18]. This metabolic arrangement has served as the foundation for theoretical characterization of efficiency and yield of methane utilization [14-15]. However, the predicted maximum carbon conversion efficiency (39-47%) was considerably less than measured values (64-66.5%) [15,19].

### SUMMARY

The technology described herein is directed to methods and compositions relating to the fermentation of methane by methantrophic microorganisms, e.g., for the production of execreted products (e.g. organic acids and/or alcohols).

In this work, multi-pronged systems-level approaches were used to reassess the metabolic functions for methane utilization in a promising bacterial biocatalyst. We demonstrate that methane assimilation is coupled with a highly efficient pyrophosphate-mediated glycolytic pathway, which under O₂ limitation participates in a novel form of fermentation-based methanotrophy. This surprising discovery suggests a novel mode of methane utilization in O₂-limited environments, and opens new opportunities for a modular approach towards producing a variety of excreted chemical products using methane as a feedstock.

In one aspect, described herein is a method for producing at least one excreted product by microbial fermentation of a gaseous substrate, comprising: (a) providing a gaseous substrate comprising CH4 and optionally, O₂, to a culture of at least one methanotrophic microorganism; and (b) maintaining the microorganism under conditions suitable for fermentation at a dissolved O₂ tension of between 0 and about 1% of saturation with air to produce at least one excreted product; or maintaining the microorganism under conditions suitable for fermentation at a dissolved O₂ tension of between 0 and about 40% of saturation with air and reducing respiration to produce at least one excreted product. In some embodiments, the methanotrophic microorganism can be a native methanotrophic microorganism. In some embodiments, reducing respiration can comprise contacting the microorganism with an inhibitor of the electron transport chain. In some embodiments, the inhibitor is antimycin A.

In some embodiments, the methanotrophic microorganism can be engineered to comprise a downregulated level of a gene selected from the group consisting of: NAD-reducing hydrogenase (MALCv4_1304 and 1307); acetate kinase (MALCv4_2853); lactate dehydrogenase (MALCv4_0534); acetate kinase (MALCv4_2853) and lactate dehydrogenase (MALCv4_0534); bacteriohemerythrin (MALCv4_2316); sucrose-phosphate synthase (MALCv4_0614); and sucrose-phosphate synthase (MALCv4_0614) and bacteriohemerythrin (MALCv4_2316); a member of the cytochrome be complex (MALCv4_0634, MALCv4_0633, and MALCv4_0632); a glycogen biosynthesis gene (MALCv4_3502; MALCv4_3503; MALCv_3504; MALCv4_3505; MALCv_3506; MALCv4_3507, and MALCv_3508); and cytochrome aa3 oxidase (MALCv4_2315). In some embodiments, the methanotrophic microorganism is engineered to comprise a mutation selected from the group consisting of: a deletion of NAD-reducing hydrogenase (MALCv4_1304 and 1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); a deletion of bacteriohemerythrin (MALCv4_2316); and a deletion of sucrose-phosphate synthase (MALCv4_0614); a deletion of sucrose-phosphate synthase (MALCv4_0614) and bacteriohemerythrin (MALCv4_2316); a deletion of a member of the cytochrome bc1 complex (MALCv4_0634, MALCv4_0633, and MALCv4_0632); a deletion of a glycogen biosynthesis gene (MALCv4_3502; MALCv4_3503; MALCv_3504; MALCv4_3505; MALCv_3506; MALCv4_3507, and MALCv_3508); and deletion of cytochrome aa3 oxidase (MALCv4_2315).

In one aspect, provided herein is a method for producing at least one excreted product by microbial fermentation of a gaseous substrate, comprising: (a) providing a gaseous substrate comprising CH₄ and optionally, O₂, to a culture of at least one methanotrophic microorganism; and (b) maintaining the microorganism under conditions suitable for fermentation to produce at least one excreted product. In some embodiments, the methanotrophic microorganism can be engineered to comprise a downregulated level of a gene selected from the group consisting of: NAD-reducing hydrogenase (MALCv4_1304 and 1307); acetate kinase (MALCv4_2853); lactate dehydrogenase (MALCv4_0534); acetate kinase (MALCv4_2853) and lactate dehydrogenase (MALCv4_0534); bacteriohemerythrin (MALCv4_2316); sucrose-phosphate synthase (MALCv4_0614); and sucrose-phosphate synthase (MALCv4_0614) and bacteriohemerythrin (MALCv4_2316); a member of the cytochrome be 1 complex (MALCv4_0634, MALCv4_0633, and MALCv4_0632); a glycogen biosynthesis gene (MALCv4_3502; MALCv4_3503; MALCv_3504; MALCv4_3505; MALCv_3506; MALCv4_3507, and MALCv_3508); and cytochrome aa3 oxidase (MALCv4_2315). In some embodiments, the methanotrophic microorganism is engineered to comprise a mutation selected from the group consisting of: a deletion of NAD-reducing hydrogenase (MALCv4_1304 and 1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); a deletion of bacteriohemerythrin (MALCv4_2316); and a deletion of sucrose-phosphate synthase (MALCv4_0614); a deletion of sucrose-phosphate synthase (MALCv4_0614) and bacteriohemerythrin (MALCv4_2316); a deletion of a member of the cytochrome be 1 complex (MALCv4_0634, MALCv4_0633, and MALCv4_0632); a deletion of a glycogen biosynthesis gene (MALCv4_3502; MALCv4_3503; MALCv_3504; MALCv4_3505; MALCv_3506; MALCv4_3507, and MALCv_3508); and deletion of cytochrome aa3 oxidase (MALCv4_2315). In some embodiments, the method can further comprise reducing respiration. In some embodiments, reducing respiration comprises contacting the microorganism with an inhibitor of the electron transport chain. In some embodiments, the inhibitor can be antimycin A. In some embodiments, the microorganism can be maintained under conditions suitable for fermentation at a dissolved O₂ tension of between 0 and about 40% of saturation with air to produce at least one excreted product.

In some embodiments of the foregoing aspects, the dissolved O₂ tension can be between 0 and about 10%. In some embodiments of the foregoing aspects, the dissolved O₂ tension can be between 0 and about 1%. In some embodiments of the foregoing aspects, the dissolved O₂ tension is between 0 and about 0.1%.

In some embodiments of the foregoing aspects, fermenting comprises: converting the gaseous substrate to intracellular formaldehyde; and converting the intracellular formaldehyde to at least one excreted product. In some embodiments, fermenting can further comprise reducing respiratory activity. In some embodiments, respiratory activity can be reduced by contacting the cell with a respiratory activity inhibitor or engineering the cell. In some embodiments, fermentation can comprise the conversion of formaldehyde to at least one excreted product by a metabolic pathway in which energy is generated by substrate-level phosphorylation.

In some embodiments of the foregoing aspects, the method can further comprise separating the at least one excreted product from the liquid nutrient media.

In some embodiments of the foregoing aspects, the culture of at least one methanotrophic microorganism can be of a genus selected from the group consisting of Methylococcus, Methylomonas, Methylomicrobium, Methylobacter, Methylomarinum, Methylovulum, Methylocaldum, Methylothermus, Methylomarinovum, Methylosphaera, Methylocystis and Methylosinus, and a mixture thereof. In some embodiments of the foregoing aspects, the culture of at least one methanotrophic microorganism can be selected from the group consisting of: Methylomicrobium alcaliphilum; Methylomicrobium buryatense; Methylomonas spp; and a mixture thereof.

In some embodiments of the foregoing aspects, the culture and liquid medium can be contained in a bioreactor. In some embodiments of the foregoing aspects, the culture and liquid medium can be contained in a closed vial.

In some embodiments of the foregoing aspects, the ratio of CH₄:O₂ in the gaseous substrate can be from about 10:1 to about 1:1. In some embodiments of the foregoing aspects, the ratio of CH₄:O₂ in the gaseous substrate can be selected from the group consisting of: about 10:1; about 5:1; about 4:1; about 2:1; about 1.5:1 and about 1:1.

In some embodiments of the foregoing aspects, the excreted product can be an organic acid. In some embodiments of the foregoing aspects, the organic acid can be selected from the group consisting of: succinate; acetate; butyrate; lactate; malate; fumarate; citrate; glycerate; formic acid; stearic acid; 3-hydroxybutyrate; propionate; and mixtures thereof.

In some embodiments of the foregoing aspects, the excreted product can be an alcohol. In some embodiments of the foregoing aspects, the alcohol can be selected from the group consisting of propanol, isopropanol, ethanol, or mixtures thereof.

In one aspect, described herein is an engineered methanotrophic bacterium capable of fermenting methane comprising a deletion of one or more of the following genes:
a. NADH-ubiquinone oxidoreductase (MALCv4_1304);
b. hydrogenase (MALCv4_1307);
c. acetate kinase (MALCv4_2853); and
d. lactate dehydrogenase (MALCv4_0534);
e. NAD-reducing hydrogenase (MALCv4_1304 and 1307);
f. bacteriohemerythrin (MALCv4_2316);
g. sucrose-phosphate synthase (MALCv4_0614);
h. a member of the cytochrome bc1 complex (MALCv4_0634, MALCv4_0633, and MALCv4_0632);
i. a glycogen biosynthesis gene (MALCv4_3502; MALCv4_3503; MALCv_3504; MALCv4_3505; MALCv_3506; MALCv4_3507, and MALCv_3508); and
j. cytochrome aa3 oxidase (MALCv4_2315).

In some embodiments, the engineered methanotrophic bacterium can be selected from the genus consisting of Methylococcus, Methylomonas, Methylomicrobium, Methylobacter, Methylothermus, Methylocaldum, Methylosphaera, Methylocystis, Methylomarinovum, Methylomicrobium alcaliphilum, Methylomicrobium buryatense and Methylosinus.

### BRIEF DESCRIPTION OF THE DRAWINGS

FIGS. 1A-1B depict an overview of methane oxidation and the ribulose-monophosphate (RuMP) pathway for formaldehyde assimilation. FIG. 1A depicts a schematic of predicted positions of incorporated ¹³CH₄-derived carbon (indicated by hexagons) for the Entner-Doudoroff (EDD) (dark arrows) and the Embden-Meyerhof-Parnas (EMP) (arrows) variants. FIG. 1B depicts summary equations for production of 3-phosphoglycerate from formaldehyde via the RuMP pathway for the EDD variant (1) or the EMP variant with either the ATP-dependent EMP pathway (2) or the PPi-dependent EMP pathway (3). The circled P indicates the phosphate (PO3)²⁻ moiety. Dashed lines indicate multistep reactions. The pentose-phosphate pathway variant for regeneration of ribulose 5-phosphate is indicated by dashed arrows. Activities of the key enzymes (in nmol min⁻¹ mg protein⁻¹) in cell free extracts of M. alcaliphilum 20Z are as follows: methane monoxygenase (Mmo), 70±5; PQQ-dependent methanol dehydrogenase (Mdh), 230±12; NAD-dependent formate dehydrogenase (Fdh), 130±7; hexulose phosphate synthase/hexulose phosphate isomerase (Hps/Hpi), 600±30; glucose phosphate isomerase (Gpi), 32±5; NADP-dependent glucose 6-phosphate dehydrogenase, 34±2 (Gpd); NAD-dependent glucose 6-phosphate dehydrogenase, 23±2 (Gdp); NADP-dependent 6-phosphogluconate dehydrogenase (Edd), 32±2; NAD-dependent 6-phosphogluconate dehydrogenase (Edd), KDPG aldolase (Eda), 60±4; 12±2; fructose-bisphosphate aldolase (Fba), 35±2; PPi-phosphofructokinase (Pfk), 70±4. Ru5P, ribulose 5-phosphate; He6P, 3-hexulose 6-phosphate; F6P, fructose 6-phosphate; KDPG, 2-keto-3-deoxy 6-phosphogluconate; F1,6P, fructose 1,6-bisphosphate; DAP, dihydroxyacetone phosphate; GAP, glyceraldehyde 3-phosphate; PGA, 3-phosphoglycerate; Pi, inorganic phosphate.

FIG. 2 demonstrates the ¹³C-pyruvate labeling patterns in M. alcaliphilum 20Z after the switch from ¹²CH₄ to ¹³CH₄. Intracellular pyruvate was resolved by multiple reactions monitoring scan mode on mass spectrometry. Depicted are: ¹³C-doubly and triply labeled pyruvate; ¹³C-pyruvate labeled in position [1]; and ¹³C-pyruvate, labeled in position [3].

FIG. 3 demonstrates ¹³C-incorporation during the switch from ¹²CH₄ to ¹³CH₄ in M. alcaliphilum 20Z. M+0 represents non-labeled compound, M+1 represents compound with one ¹³C-label, M+2 represents compound with two ¹³C-labels, etc. F6P/G6P, fructose-6-phosphate+glucose-6-phosphate; PEP, phosphoenolpyruvate; 2PG/3PG, 2-phosphoglycerate+3-phosphoglycerate. All experiments were performed in triplicates. Error bars represent standard deviation.

FIG. 4 demonstrates ¹³C-pyruvate labeling patterns in Methylomonas sp. LW13, after the switch from 12CH₄ to ¹³CH₄. Intracellular pyruvate was resolved by multiple reactions monitoring scan mode on mass spectrometry. Depicted are: ¹³C-doubly and triply labeled pyruvate; ¹³C-pyruvate labeled in position [1]; and ¹³C-pyruvate, labeled in position [3].

FIG. 5 depicts predicted pathways for mixed acid fermentation and H₂ production in M. alcaliphilum 20Z.

FIG. 6 depicts the sequence of the genetic construction for cytochrome bc1 and ubiquinol-cytochrome c reductase complex (complex III) (SEQ ID NO: 1). Gene deletion region is shown in italics, primers are shown in bold font; start and stop codons are underlined.

FIG. 7 depicts the sequence of the genetic construction for acetate kinase (SEQ ID NO: 2). Gene deletion region is shown in italics, primers are shown in bold font; start and stop codons are underlined.

FIG. 8 depicts the sequence of the genetic construction for HOX NAD-reducing hydrogenase hoxS subunit beta (SEQ ID NO: 3). Gene deletion region is shown in italics, primers are shown in bold font; start and stop codons are underlined.

FIG. 9 depicts the sequence of the genetic construction for NAD-reducing hydrogenase hoxS subunit alpha (SEQ ID NO: 4). Gene deletion region is shown in italics, primers are shown in bold font; start and stop codons are underlined.

FIG. 10 depicts the sequence of the genetic construction for lactate dehydrogenase (SEQ ID NO: 5). Gene deletion region is shown in italics, primers are shown in bold font; start and stop codons are underlined.

FIG. 11 depicts the sequence of the genetic construction for Na(+)-translocating NADH-quinone reductase subunit A (SEQ ID NO: 6). Gene deletion region is shown in italics, primers are shown in bold font; start and stop codons are underlined.

### DETAILED DESCRIPTION

As described herein, the inventors have discovered that methantrophic microorganisms can ferment methane to produce excreted products. The inventors have further identified mutations and fermentation conditions (e.g. oxygen levels) that can provide improved yields of one or more of the desired products. Accordingly, provided herein are methods and compositions relating to the production of excreted products by microbial fermentation of methane.

The conditions and methods described herein differ significantly from any naturally-occurring conditions, e.g., the monoculture (or nearly monoculture) nature of culturing bacteria in a non-natural environment, a reduced accumulation of end products (e.g., excreted products) due to culture flow which can reduce negative feedback loops, a lack of limiting nutrients, a lack of competition and/or inhibition due to competing organisms, a steady supply or level of nutrients, temperature, pH, methane, oxygen, etc. which can provide continuous activity of a given metabolic pathway as opposed to the constant adaptation necessitated by natural environments, and the like.

In one aspect, provided herein is a method for producing at least one excreted product by microbial fermentation of a gaseous substrate, comprising: providing a gaseous substrate comprising CH₄ and optionally, O₂, to a culture of at least one methanotrophic microorganism in a liquid nutrient medium; and maintaining the microorganism under conditions suitable for fermentation at a dissolved O₂ tension of between 0 and about 1% of saturation with air to produce at least one excreted product. In some embodiments, the methanotrophic microorganism is a native methanotrophic microorganism.

In one aspect, provided herein is a method for producing at least one excreted product by microbial fermentation of a gaseous substrate, comprising: providing a gaseous substrate comprising CH₄ and optionally, O₂, to a culture of at least one methanotrophic microorganism in a liquid nutrient medium; and maintaining the microorganism under conditions suitable for fermentation to produce at least one excreted product.

In some embodiments, the methantrophic microorganism can be engineered to comprise a downregulated level of a gene selected from the group consisting of: NAD-reducing hydrogenase (MALCv4_1304 and 1307); acetate kinase (MALCv4_2853); lactate dehydrogenase (MALCv4_0534); acetate kinase (MALCv4_2853) and lactate dehydrogenase (MALCv4_0534); bactcriohemerythrin (MALCv4_2316); sucrose-phosphate synthase (MALCv4_0614); sucrose-phosphate synthase (MALCv4_0614) and bacteriohemerythrin (MALCv4_2316); a member of the cytochrome bc1 complex (MALCv4_0634, MALCv4_0633, and MALCv4_0632); a glycogen biosynthesis gene (MALCv4_3502; MALCv4_3503; MALCv_3504; MALCv4_3505; MALCv_3506; MALCv4_3507, and MALCv_3508); and cytochrome aa3 oxidase (MALCv4_2315). In some embodiments, the methantrophic microorganism can be engineered to comprise a mutation selected from the group consisting of: a deletion of NAD-reducing hydrogenase (MALCv4_1304 and 1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); a deletion of bacteriohemerythrin (MALCv4_2316); and a deletion of sucrose-phosphate synthase (MALCv4_0614); a deletion of sucrose-phosphate synthase (MALCv4_0614) and bacteriohemerythrin (MALCv4_2316); a deletion of a member of the cytochrome bc1 complex (MALCv4_0634, MALCv4_0633, and MALCv4_0632); a deletion of a glycogen biosynthesis gene (MALCv4_3502; MALCv4_3503; MALCv_3504; MALCv4_3505; MALCv_3506; MALCv4_3507, and MALCv_3508); and a deletion of cytochrome aa3 oxidase (MALCv4_2315).

In some embodiments, the methanotrophic microorganism can be engineered to comprise a downregulated level of a gene selected from the group consisting of: NADH-ubiquinone oxidoreductase (MALCv4_2233); hydrogenase (MALCv4_1304-MALCv4_1307); acetate kinase (MALCv4_2853); lactate dehydrogenase (MALCv4_0534); acetate kinase (MALCv4_2853) and lactate dehydrogenase (MALCv4_0534); and cytochrome bc1 complex (MALCv4_0634, MALCv4_0633 and MALCv4_0632 genes). In some embodiments, the methanotrophic microorganism can be engineered to comprise a mutation selected from the group consisting of: a deletion of NADH-ubiquinone oxidoreductase (MALCv4_2233); a deletion of hydrogenase (MALCv4_1304-MALCv4_1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); and a deletion of cytochrome be 1 complex (MALCv4_0634, MALCv4_0633 and MALCv4_0632 genes).

As described herein, “fermentation” refers to an activity or process involving enzymatic or metabolic decomposition (digestion) of organic materials by microorganisms. The fermentation process can also involve production of useful compounds and substances, typically organic compounds and substances, by the microorganisms. In some embodiments, fermenting can comprise converting the gaseous substrate to intracellular formaldehyde; and converting the intracellular formaldehyde to at least one excreted product. In some embodiments, fermentation can comprise the conversion of formaldehyde to at least one excreted product by a metabolic pathway in which energy is generated by substrate-level phosphorylation.

In some embodiments, respiratory activity is reduced during the fermentation step. Means of reducing respiratory activity of microorganisms are known in the art and can include, by way of non-limiting example, contacting the cell with a respiratory activity inhibitor or engineering the cell. For example, inhibiting the electron transport chain, e.g. by downregulating, mutating, and/or deleting a gene involved in the electron transport chain (e.g. complex I, complex II, complex III, complex IV, and ATP synthase) or by contacting the cell with an inhibitor of the electron transport chain (e.g., antimycin, antimycin A, Amytal, Rotenone, Sodium Azide, Demerol, 2-thenoyltrifluoroacetone, carboxin, DCCD, oligomycin and Cyanides) can reduce respiration. In some embodiments, respiratory activity can be reduced by contacting the cell with an inhibitor of the electron transport chain. For further description of the inhibition of respiration, see, e.g., Lim. Microbiology. Kendall Hunt (2003); and Zannoni. Respiration in Archaea and Bacteria. Springer Science (2004); each of which is incorporated by reference herein in its entirety.

In some embodiments, the microorganism can be maintained under conditions suitable for fermentation at a dissolved O₂ tension of between 0 and about 50% of saturation with air to produce at least one excreted product. In some embodiments, the microorganism can be maintained under conditions suitable for fermentation at a dissolved O₂ tension of between 0 and about 40% of saturation with air to produce at least one excreted product. In some embodiments, the microorganism can be maintained under conditions suitable for fermentation at a dissolved O₂ tension of between 0 and about 20% of saturation with air to produce at least one excreted product. In some embodiments, the microorganism can be maintained under conditions suitable for fermentation at a dissolved O₂ tension of between 0 and about 10% of saturation with air to produce at least one excreted product. In some embodiments, the microorganism can be maintained under conditions suitable for fermentation at a dissolved O₂ tension of between 0 and about 1% of saturation with air to produce at least one excreted product. In some embodiments, the microorganism can be maintained under conditions suitable for fermentation at a dissolved O₂ tension of between 0 and about 0.1% of saturation with air to produce at least one excreted product. In some embodiments, a native microorganism can be maintained under conditions suitable for fermentation at a dissolved O₂ tension of between 0 and about 1% of saturation with air to produce at least one excreted product. In some embodiments, a native microorganism can be maintained under conditions suitable for fermentation at a dissolved O₂ tension of between 0 and about 0.1% of saturation with air to produce at least one excreted product.

In some embodiments, the ratio of CH₄:O₂ in the gaseous substrate can be from about 20:1 to about 1:2. In some embodiments, the ratio of CH₄:O₂ in the gaseous substrate can be from 20:1 to 1:2. In some embodiments, the ratio of CH₄:O₂ in the gaseous substrate can be from about 10:1 to about 1:1. In some embodiments, the ratio of CH₄:O₂ in the gaseous substrate can be from 10:1 to 1:1. In some embodiments, the ratio of CH₄:O₂ in the gaseous substrate can be selected from the group consisting of: about 10:1; about 5:1; about 4:1; about 2:1; about 1.5:1 and about 1:1. In some embodiments, the ratio of CH₄:O₂ in the gaseous substrate can be selected from the group consisting of: 10:1; 5:1; 4:1; 2:1; 1.5:1 and 1:1.

In some embodiments, the microorganism can be cultured and/or maintained in a liquid medium. In some embodiments, the microorganism can be cultured and/or maintained on a substrate, e.g. beads, filters, fluidics devices, microfluidics devices, membranes and the like. Microorganisms can be bound to a substrate, e.g. by providing a substrate that can bind to a molecule displayed on the surface of the microorganism, by causing the microorganism to express a molecule (e.g. a protein) that can bind the substrate (e.g., either a native or engineered molecule), and/or by allowing the microorganism to colonize the substrate (e.g. biofilm formation and/or settling out of suspension. In some embodiments, liquid medium can be provided to the substrate.

The excreted products produced in accordance with the methods described herein can comprise a carbon-containing compound which is excreted by the microbial cell into the extracellular medium, e.g. either by active or passive transport across the cellular membrane. The excreted product can be, e.g. an organic acid and/or an alcohol. Non-limiting examples of organic acids can include succinate, acetate, butyrate, lactate, malate, fumarate, citrate, glycerate, formic acid, stearic acid, 3-hydroxybutyrate, and propionate, or mixtures thereof. Non-limiting examples of alcohols can include propanol, isopropanol, ethanol, butanediol, or mixtures thereof. In some embodiments, the alcohol can be propanol, isopropanol, ethanol, or mixtures thereof.

As described herein, “methanotrophic microorganism” refers to are bacteria that are able to metabolize methane as their only source of carbon. Non-limiting examples of methantrophic microorganisms can include Methylomicrobium alcaliphilum or Methylomicrobium buryatense, Methylomonas spp, or mixtures thereof and/or the genus consisting of Methylococcus, Methylomonas, Methylomicrobium, Methylobacter, Methylomarinum, Methylovulum, Methylocaldum, Methylothermus, Methylomarinovum, Methylosphaera, Methylocystis and Methylosinus, or mixtures there of. In the methods described herein, a single type of methantrophic microorganism or a combination of types of methantrophic microorganisms can be used. In some embodiments, the methantrophic microorganism can be Methylomicrobium buryatense strain 5 GB1 (“5 GB1”) and/or Methylomicrobium alcaliphilum strain 20Z (“20Z”). By way of non-limiting example, the method can be performed with two species or with two different mutant strains of methantrophic microorganisms.

Methanotrophs are a highly specialized bacterial group utilizing methane (CH₄) as a sole source of carbon and energy. Obligate aerobic MB can be separated into three major groups. Group I MB are gammaproteobacteria that have stacked membranes built mostly of C16 fatty acids. Group I MB use the ribulose monophosphate (RuMP) cycle, which converts formaldehyde (CH₂OH) into multi-carbon compounds for building cell biomass. The majority of Group I methanotrophs are grouped into the Methylococcaceae family. Group II MB are alphaproteobacteria, contain rings of particulate methane monooxygenase (pMMO)-harboring membranes at the cell periphery, generally accumulate C18 fatty acids, and use the serine cycle for converting formaldehyde into biomass. Methylocystis and Methylosinus species are typical representatives of Group II MB. Group III MB do not produce intracellular membranes (ICM), display a low growth rate, and assimilate carbon through the Calvin-Benson-Bassham (CBB) cycle. Group III MP are represented by methanotrophic Verrucomicrobia. MB are unique in their ability to synthesize lipids from methane. Group I MB particularly have relatively high lipid/biomass content (as high as 22% total lipid in 5 GB1) as a result of formation of extensive intracellular membranes (ICM). Accordingly, Group I MB are the focus of the current disclosure.

In some embodiments, the methantrophic microorganism can be a native or wildtype microorganism, e.g. a microorganism that is not engineered. In some embodiments, the methanotrophic microorganism can be a naturally-occurring variant or mutant, e.g. one that is selected for comprising a modulation and/or mutation (as compared to wild-type) as described herein.

In some embodiments, the methantrophic microorganism can be an engineered microorganism. As used herein, “engineered” refers to the aspect of having been manipulated by the hand of man. For example, in some embodiments of the present invention, an engineered bacterium comprises an engineered polynucleotide, e.g., comprises a mutation resulting in a polynucleotide sequence not found in nature. As is common practice and is understood by those in the art, progeny and copies of an engineered polynucleotide are typically still referred to as “engineered” even though the actual manipulation was performed on a prior entity. As used herein, “mutation” or “genetic alteration” refers to a change or difference in the genetic material of a cell as compared to a reference wildtype cell, e.g. a deletion, an insertion, a SNP, a gene rearrangement, a mutation, and/or the introduction of an exogenous gene or sequence.

As used herein “enhanced activity” can refer to an upregulation of the expression and/or activity of the gene in question. As used herein, “up-regulation” or “up-regulated” means increasing an activity within a bacterial cell. The activity can be the actions of one or more metabolic pathways or portions of metabolic pathways within a bacterial cell. An up-regulation of one activity can be caused by the down-regulation of another. Alternatively, an up-regulation of an activity can occur through increased activity of an intracellular protein, increased potency of an intracellular protein or increased expression of an intracellular protein. The protein with increased activity, potency or expression can be encoded by genes disclosed herein.

To cause an up-regulation through increased expression of a protein, the copy number of a gene or genes encoding the protein may be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as necessary for the methods and compositions described herein.

As used herein, “down-regulation” or “down-regulated” means any action at the metabolic pathway, protein or gene level that results in: a decrease in the activity of a metabolic pathway or a portion thereof; a decrease in activity of a protein; elimination of a protein's activity, translation of an incomplete protein sequence; incorrect folding of protein; reduced transcription of a gene; incomplete transcription of a gene, interference with an encoded RNA transcript, or any other activity resulting in reduced activity of a pathway, protein or gene. An increase in the expression of a pathway inhibitory protein or signaling molecule can also result in pathway downregulation.

A gene can be down-regulated for example by insertion of a foreign set of base pairs in a coding region, deletion of a portion of the gene, or by the presence of antisense sequences that interfere with transcription or translation of the gene. In another embodiment, down-regulation includes elimination of a gene's expression (i.e. gene knockout). As used herein, the symbol “Δ” denotes a mutation in the specified coding sequence and/or promoter wherein at least a portion (up to and including all) of the coding sequence and/or promoter has been disrupted by a deletion, mutation, or insertion. In another embodiment, the disruption can occur by optionally inserting a nucleotide or polynucleotide molecule into the native gene sequence whereby the expression of the mutated gene is down-regulated (either partially or completely). Any one or a combination of these approaches can be used to effect downregulation of a desired target protein as necessary for the methods and compositions described herein.

As used herein, “deletion”, when used to refer to the deletion of a given gene refers to a mutation (e.g. natural and/or engineered) that reduces the expression and/or activity of the polypeptide gene product by 90% or more, e.g., 95% or more, 99% or more. A deletion can comprise, by way of non-limiting example, a mutation of regulatory sequences and/or coding sequences. In some embodiments, a deletion can comprise a deletion of the coding sequence of a gene, e.g., 80% or more, 85% or more, 90% or more, or 95% or more of the coding sequence of the gene.

“Up-regulation” and “down-regulation” can be measured against a control condition including, without limitation, relative to the activity of an unmodified bacterial strain of the same species.

As used herein, “heterologously expressing” when used in reference to a cell refers to a cell which is expressing a detectable level of a heterolog of a native gene.

In some embodiments, 4-hydroxybutyrate reductase can refer to carboxylic acid reductase, an exemplary example of which is E. coli carboxylic acid reductase (e.g., NCBI Gene ID: 7149022).

As used herein, “maintaining” refers to continuing the viability of a cell or population of cells. A maintained population of cells will have at least a subpopulation of metabolically active cells.

As used herein, “conditions suitable for fermentation” refers to conditions under which a deteactable level of fermentation occurs. Such conditions can comprise those under which a bacterium as described herein is metabolically active and provided access to methane. Examples of suitable conditions are provided, e.g., in the Examples herein.

In some embodiments, the method can further comprise separating and/or isolating the at least one excreted product from the liquid nutrient media. Methods of separating excretion products are known in the art and can include, by way of non-limiting example, centrifugation, phase separation, filters, affinity columns or matrices, distillation,

In some embodiments, the culture and liquid medium can be contained in a bioreactor. In some embodiments, the culture and liquid medium can be contained in a closed vial.

In one aspect, described herein is an engineered methanotrophic bacterium capable of fermenting methane comprising a deletion of one or more of the following genes:
a. NADH-ubiquinone oxidoreductase (MALCv4_2233),
b. hydrogenase (MALCv4_1304-MALCv4_1307),
c. acetate kinase (MALCv4_2853),
d. lactate dehydrogenase (MALCv4_0534), and
e. cytochrome bc1 complex (MALCv4_0634, MALCv4_0633 and MALCv4_0632).

In one aspect, described herein is an engineered methantrophic bacterium capable of fermenting methane comprising a deletion of one or more of the following genes:
a. NADH-ubiquinone oxidoreductase (MALCv4_1304);
b. hydrogenase (MALCv4_1307);
c. acetate kinase (MALCv4_2853); and
d. lactate dehydrogenase (MALCv4_0534).

In one aspect, described herein is an engineered methanotrophic bacterium with enhanced activity of one or more of the following genes and/or heterologously expressing one or more of the following genes:
a. alcohol dehydrogenase Ald (EC 1.1.1.1),
b. alcohol dehydrogenase AldE (EC 4.1.1.1),
c. acetoacetate decarboxylase (EC 4.1.1.4),
d. 4-hydroxybutyrate dehydrogenase (EC 1.1.1.61),
e. 4-hydroxybutyrate CoA-transferase (AbfT),
f. 4-hydroxybutyrate reductase (Hbr), and
g. glutamate decarboxylase-4-aminobutanoate (EC 4.1.1.15).

TABLE 11

| | Exemplary | Exemplary |
|---|---|---|
| | Gene/Gene | SEQ ID |
| Gene(or protein complex) Name | Product | NO: |
| NADH-ubiquinone oxioreductase | MALCv4_2233 | |
| hydrogenase | MALCv4_1304- | 3-4 |
| | MALCv4_1307 | |
| Acetate kinase | MALCv4_2853 | 2 |
| Lactate dehydrogenase | MALCv4_0534 | 5 |
| Cytochrome bc1 complex | MALCv4_0632- | 1 |
| | MALCv4_0634 | |
| NADH-ubiquinone oxioreductase | MALCv4_1304 | 6 |
| Glycogen biosynthesis gene | MALCv4_3502- | |
| | 3508 | |
| Cytochrome aa3 oxidase | MALCv4_2315 | |
| Alcohol dehydrogenase (Ald) | EC 1.1.1.1 | |
| Alcohol dehydrogenase (AldE) | EC 4.1.1.1 | |
| Acetoacetate decarboxylase | EC 4.1.1.4 | |
| 4-hydroxybutyrate dehydrogenase | EC 1.1.1.61 | |
| 4-hydroxybutyrate CoA-transferase (AbfT) | NCBI Gene ID | |
| | 8469452 | |
| 4-hydroxybutyrate reductase (Hbr) | | |
| glutamate decarboxylase-4- | EC 41.1.15 | |
| aminobutanoate | | |
| bacteriohemerythrin | MALCv4_2316 | 8 |
| sucrose-phosphate synthase | MALCv4_0614 | |

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

In the present disclosure, we analyze the understanding of metabolic functions essential for methane utilization through detailed investigations of C₁-assimilation in Methylomicrobium alcaliphilum strain 20Z, a haloalkalitolerant methanotroph that is a promising biocatalyst [11]. Availability of the M. alcaliphilum 20Z genome sequence [21] allowed us to apply systems-level approaches including genome-wide transcriptomic studies (Illumina-based RNA-Seq), metabolomics, and ¹³C-label distribution analysis of methane-grown cultures for metabolic reconstruction of C₁ utilization pathways.

Additionally, in the present disclosure, we provide methods of producing at least one excreted product by microbial fermentation of a gaseous substrate using methanotrophs.

Some embodiments of the technology described herein can be defined according to any of the following numbered paragraphs:
1. A method for producing at least one excreted product by microbial fermentation of a gaseous substrate, comprising:
   a. providing a gaseous substrate comprising CH₄ and O₂ to a culture of at least one methanotrophic microorganism in a liquid nutrient medium; and
   b. fermenting the gaseous substrate at a dissolved O₂ tension of between 0 and 1% of saturation with air to produce at least one excreted product.
2. The method of paragraph 1, wherein fermenting comprises:
   a. converting the gaseous substrate to intracellular formaldehyde; and
   b. converting the intracellular formaldehyde to at least one excreted product.
3. The method of paragraph 1, wherein fermentation comprises the conversion of formaldehyde to at least one excreted product by a metabolic pathway in which energy is generated by substrate-level phosphorylation.
4. The method of paragraph 1, further comprising separating the at least one excreted product from the liquid nutrient media.
5. The method of paragraph 1, wherein the culture of at least one methanotrophic microorganism is selected from the genus consisting of Methylococcus, Methylomonas, Methylomicrobium, Methylobacter, Methylomarinum, Methylovulum, Methylocaldum, Methylosphaera, Methylocystis and Methylosinus, or mixtures there of.
6. The process of paragraph 1, wherein the culture of at least one methanotrophic microorganism is selected from Methylomicrobium alcaliphilum or Methylomicrobium buryatense, or mixtures thereof.
7. The method of paragraph 1, wherein the methanotrophic microorganism is a naturally occurring species.
8. The method of paragraph 1, wherein the methanotrophic microorganism is an engineered species.
9. The method of paragraph 8, wherein the engineered methanotrophic organism has a deletion of NADH-ubiquinone oxidoreductase (MALCv4_2233).
10. The method of paragraph 8, wherein the engineered methanotrophic organism has a deletion of hydrogenase (MALCv4_1304-MALCv4_1307).
11. The method of paragraph 8, wherein the engineered methanotrophic organism has a deletion of acetate kinase (MALCv4_2853) and lactate dehydrogenase (MALCv4_0534).
12. The method of paragraph 8, wherein the engineered methanotrophic organism has a deletion of cytochrome bc1 complex (MALCv4_0634, MALCv4_0633 and MALCv4_0632 genes).
13. The method of paragraph 1, wherein the culture and liquid medium are contained in a bioreactor.
14. The method of paragraph 1, wherein the culture and liquid medium are contained in a closed vial.
15. The method of paragraph 1, wherein the ratio of CH₄:O₂ in the gaseous substrate is between 10:1; 5:1, 4:1; 2:1, or 1:1.
16. The method of paragraph 1, wherein the excreted product is an organic acid.
17. The method of paragraph 1, wherein the excreted product is an alcohol.
18. The method of paragraph 16, wherein the organic acid is selected from the group consisting of succinate, acetate, butyrate, lactate, malate, fumarate, citrate, formic acid, stearic acid, 3-hydroxybutyrate, and propionate, or mixtures thereof.
19. The method of paragraph 16, wherein the alcohol is selected from the group consisting of propanol, isopropanol, ethanol, butanediol, or mixtures thereof
20. An engineered methanotrophic bacteria capable of fermenting methane comprising a deletion of one or more of the following genes:
   a. NADH-ubiquinone oxidoreductase (MALCv4_2233),
   b. hydrogenase (MALCv4_1304-MALCv4_1307),
   c. acetate kinase (MALCv4_2853),
   d. lactate dehydrogenase (MALCv4_0534), and
   e. cytochrome bc1 complex (MALCv4_0634, MALCv4_0633 and MALCv4_0632).
21. An engineered methanotrophic bacteria with enhanced activity of one or more of the following genes and/or heterologously expressing of one or more of the following genes:
   a. alcohol dehydrogenase Ald (EC 1.1.1.1),
   b. alcohol dehydrogenase AldE (EC 4.1.1.1),
   c. acetoacetate decarboxylase (EC 4.1.1.4),
   d. 4-hydroxybutyrate dehydrogenase (EC 1.1.1.61),
   e. 4-hydroxybutyrate CoA-transferase (AbfT),
   f. 4-hydroxybutyrate reductase (Hbr), and
   g. glutamate decarboxylase-4-aminobutanoate (EC 4.1.1.15).
22. The engineered methanotrophic bacteria of paragraphs 20 or 21, wherein the engineered methanotrophic bacteria are selected from the genus consisting of Methylococcus, Methylomonas, Methylomicrobium, Methylobacter, Methylocaldum, Methylosphaera, Methylocystis, Methylomicrobium alcaliphilum, Methylomicrobium buryatense and Methylosinus.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The term “gaseous substrate” includes any gas which contains a compound or element used by a microorganism as a carbon source and optionally energy source in microbial conversion. The gaseous substrate will typically contain a significant proportion of CH₄ and air and O₂. Similarly, the term “substrate” includes any gas and/or liquid which contains a compound or element used by a microorganism as a carbon source and optionally energy source in microbial conversion. Examples of liquid substrates include methanol.

The term “reactor” and/or “bioreactor” includes any microbial conversion device consisting of one or more vessels and/or towers or piping arrangements, such as an immobilised cell reactor, a gas-lift reactor, a bubble column reactor (BCR), a circulated loop reactor, a membrane reactor, such as a Hollow Fibre Membrane Bioreactor (HFM BR) or a trickle bed reactor (TBR).

The term “liquid nutrient media”, “media” and/or “medium” includes a liquid medium comprising nutrients suitable for microbial conversion using one or more microorganisms. The liquid nutrient media will contain vitamins and/or minerals sufficient to permit growth of the micro-organism(s) used.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker, an “increase” is a statistically significant increase in the level of such a marker.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “exogenous” refers to a substance present in a cell other than its native source. The term “exogenous” when used herein can refer to a nucleic acid or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in low amounts and one wishes to increase the amount of the nucleic acid or polypeptide in the cell or organism. A substance will be considered exogenous if it is introduced into a cell or an ancestor of the cell from which the cell has inherited the substance. In contrast, the term “endogenous” refers to a substance that is native to the biological system or cell (e.g. the microbial cell and/or target cell). As used herein, “ectopic” refers to a substance that is found in an unusual location and/or amount. An ectopic substance can be one that is normally found in a given cell, but at a much lower amount and/or at a different time.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method for producing at least one excreted product by microbial fermentation of a gaseous substrate, comprising:
   a. providing a gaseous substrate comprising CH4 and optionally, 02, to a culture of at least one methanotrophic microorganism; and
   b. maintaining the microorganism under conditions suitable for fermentation at a dissolved O₂ tension of between 0 and about 1% of saturation with air to produce at least one excreted product; or maintaining the microorganism under conditions suitable for fermentation at a dissolved O₂ tension of between 0 and about 40% of saturation with air and reducing respiration to produce at least one excreted product.
2. The method of paragraph 1, wherein the methanotrophic microorganism is a native methanotrophic microorganism.
3. The method of any of paragraphs 1-2, wherein reducing respiration comprises contacting the microorganism with an inhibitor of the electron transport chain.
4. The method of paragraph 3, wherein the inhibitor is antimycin A.
5. A method for producing at least one excreted product by microbial fermentation of a gaseous substrate, comprising:
   a. providing a gaseous substrate comprising CH4 and optionally, O₂, to a culture of at least one methanotrophic microorganism; and
   b. maintaining the microorganism under conditions suitable for fermentation to produce at least one excreted product.
6. The method of paragraph 5, wherein the methanotrophic microorganism is engineered to comprise a downregulated level of a gene selected from the group consisting of:
   NAD-reducing hydrogenase (MALCv4_1304 and 1307); acetate kinase (MALCv4_2853); lactate dehydrogenase (MALCv4_0534); acetate kinase (MALCv4_2853) and lactate dehydrogenase (MALCv4_0534); bacteriohemerythrin (MALCv4_2316); sucrose-phosphate synthase (MALCv4_0614); and sucrose-phosphate synthase (MALCv4_0614) and bacteriohemerythrin (MALCv4_2316); a member of the cytochrome be 1 complex (MALCv4_0634, MALCv4_0633, and MALCv4_0632); a glycogen biosynthesis gene (MALCv4_3502; MALCv4_3503; MALCv_3504; MALCv4_3505; MALCv_3506; MALCv4_3507, and MALCv_3508); and cytochrome aa3 oxidase (MALCv4_2315).
7. The method of paragraph 6, wherein the methanotrophic microorganism is engineered to comprise a mutation selected from the group consisting of:
   a deletion of NAD-reducing hydrogenase (MALCv4_1304 and 1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); a deletion of bacteriohemerythrin (MALCv4_2316); and a deletion of sucrose-phosphate synthase (MALCv4_0614); a deletion of sucrose-phosphate synthase (MALCv4_0614) and bacteriohemerythrin (MALCv4_2316); a deletion of a member of the cytochrome bc1 complex (MALCv4_0634, MALCv4_0633, and MALCv4_0632); a deletion of a glycogen biosynthesis gene (MALCv4_3502; MALCv4_3503; MALCv_3504; MALCv4_3505; MALCv_3506; MALCv4_3507, and MALCv_3508); and deletion of cytochrome aa3 oxidase (MALCv4_2315).
8. The method of any of paragraphs 5-7, wherein the method further comprises reducing respiration.
9. The method of paragraph 8, wherein reducing respiration comprises contacting the microorganism with an inhibitor of the electron transport chain.
10. The method of paragraph 9, wherein the inhibitor is antimycin A.
11. The method of any of paragraphs 5-10, wherein the microorganism is maintained under conditions suitable for fermentation at a dissolved O₂ tension of between 0 and about 40% of saturation with air to produce at least one excreted product.
12. The method of any of paragraphs 5-11, wherein the dissolved O₂ tension is between 0 and about 10%.
13. The method of any of paragraphs 5-12, wherein the dissolved O₂ tension is between 0 and about 1%.
14. The method of any of paragraphs 1-13, wherein the dissolved O₂ tension is between 0 and about 0.1%.
15. The method of any of paragraphs 1-4, wherein fermenting comprises:
   a. converting the gaseous substrate to intracellular formaldehyde; and
   b. converting the intracellular formaldehyde to at least one excreted product.
16. The method of paragraph 15, wherein fermenting further comprises reducing respiratory activity.
17. The method of paragraph 16, wherein respiratory activity is reduced by contacting the cell with a respiratory activity inhibitor or engineering the cell.
18. The method of any of paragraphs 1-17, wherein fermentation comprises the conversion of formaldehyde to at least one excreted product by a metabolic pathway in which energy is generated by substrate-level phosphorylation.
19. The method of any of paragraphs 1-18, further comprising separating the at least one excreted product from the liquid nutrient media.
20. The method of any of paragraphs 1-19, wherein the culture of at least one methanotrophic microorganism is of a genus selected from the group consisting of Methylococcus, Methylomonas, Methylomicrobium, Methylobacter, Methylomarinum, Methylovulum, Methylocaldum, Methylothermus, Methylomarinovum, Methylosphaera, Methylocystis and Methylosinus, and a mixture thereof.
21. The method of any of paragraphs 1-20, wherein the culture of at least one methanotrophic microorganism is selected from the group consisting of: Methylomicrobium alcaliphilum; Methylomicrobium buryatense; Methylomonas spp; and a mixture thereof.
22. The method of any of paragraphs 1-21, wherein the culture and liquid medium are contained in a bioreactor.
23. The method of any of paragraphs 1-22, wherein the culture and liquid medium are contained in a closed vial.
24. The method of any of paragraphs 1-23, wherein the ratio of CH₄:O₂ in the gaseous substrate is from about 10:1 to about 1:1
25. The method of paragraph 24, wherein the ratio of CH₄:O₂ in the gaseous substrate is selected from the group consisting of:
   about 10:1; about 5:1; about 4:1; about 2:1; about 1.5:1 and about 1:1.
26. The method of any of paragraphs 1-25, wherein the excreted product is an organic acid.
27. The method of any of paragraphs 1-26, wherein the excreted product is an alcohol.
28. The method of paragraph 27, wherein the organic acid is selected from the group consisting of:
   succinate; acetate; butyrate; lactate; malate; fumarate; citrate; glycerate; formic acid; stearic acid; 3-hydroxybutyrate; propionate; and mixtures thereof.
29. The method of paragraph 27, wherein the alcohol is selected from the group consisting of propanol, isopropanol, ethanol, or mixtures thereof.
30. An engineered methanotrophic bacterium capable of fermenting methane comprising a deletion of one or more of the following genes:
   a. NADH-ubiquinone oxidoreductase (MALCv4_1304);
   b. hydrogenase (MALCv4_1307);
   c. acetate kinase (MALCv4_2853); and
   d. lactate dehydrogenase (MALCv4_0534);
   e. NAD-reducing hydrogenase (MALCv4_1304 and 1307);
   f. bacteriohemerythrin (MALCv4_2316);
   g. sucrose-phosphate synthase (MALCv4_0614);
   h. a member of the cytochrome bc1 complex (MALCv4_0634, MALCv4_0633, and MALCv4_0632);
   i. a glycogen biosynthesis gene (MALCv4_3502; MALCv4_3503; MALCv_3504; MALCv4_3505; MALCv_3506; MALCv4_3507, and MALCv_3508); and
j. cytochrome aa3 oxidase (MALCv4_2315).
31. The engineered methanotrophic bacterium of paragraph 30, wherein the engineered methanotrophic bacterium is selected from the genus consisting of Methylococcus, Methylomonas, Methylomicrobium, Methylobacter, Methylothermus, Methylocaldum, Methylosphaera, Methylocystis, Methylomarinovum, Methylomicrobium alcaliphilum, Methylomicrobium buryatense and Methylosinus.

### EXAMPLES

### Example 1

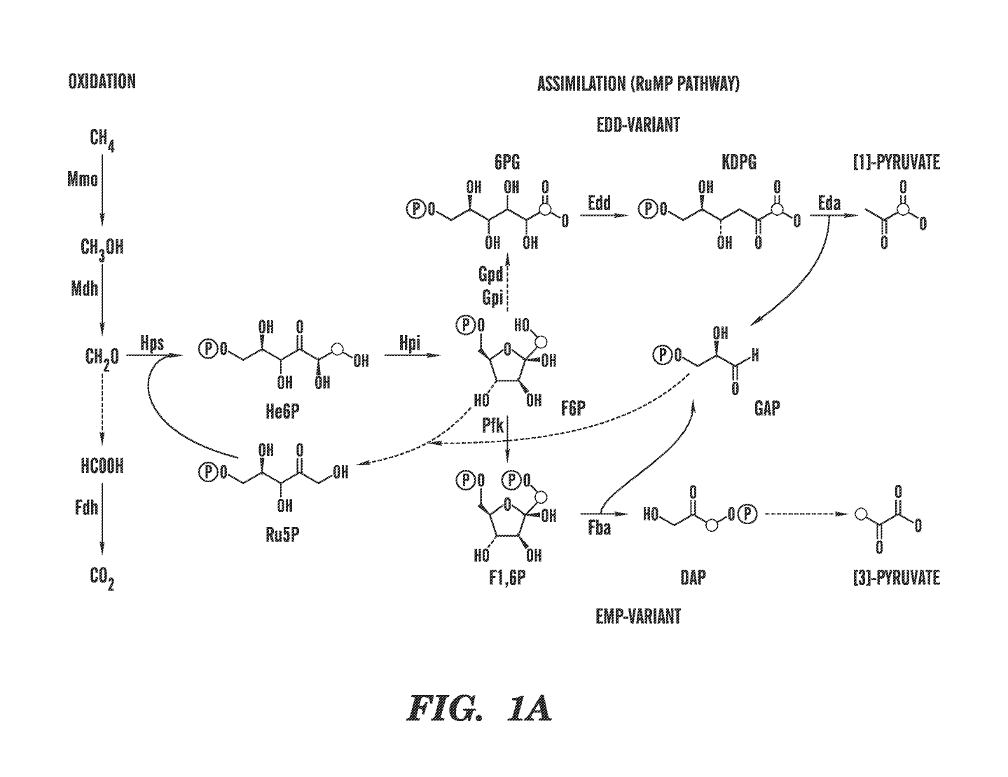

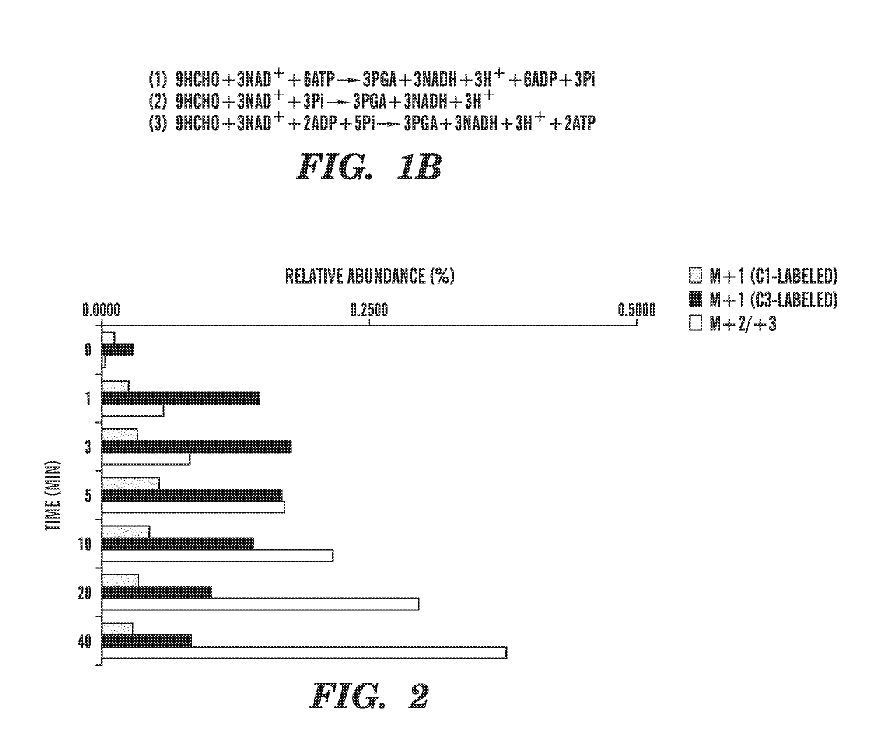

Transcriptomic study. M. alcaliphilum 20Z grown aerobically with methane as the sole source of carbon and energy showed high levels of expression for genes known to be involved in the metabolism of C₁ compounds including those for membrane-bound methane monooxygenase (pmoCAB), PQQ-dependent methanol dehydrogenase (mxaFIG), and two key enzymes of the RuMP pathway-hexulose phosphate synthase (hps) and phosphohexuloisomerase (hpi) (FIG. 1A, Table 3), as expected. The relative abundance of transcripts for enzymes involved in the RuMP pathway downstream from fructose-6-phosphate were 3-20 fold lower than those of hpi and hps. Remarkably the abundance of transcripts encoding glycolytic pathway enzymes was 2-10 fold higher than EDD pathway enzymes (i.e., edd and eda) (Table 3). Furthermore, one of the putative pyruvate kinase genes, pyk2 (MALCv4_3080), showed high expression.

Methanotrophic pyruvate kinase-purification and characterization. In accordance with previous studies, no pyruvate-forming activity was detected in cell-free extracts of Methylomicrobium alcaliphilum strain 20Z with three different enzymatic assays (see materials and methods). However, when the pyruvate kinase gene was overexpressed in E. coli, purified protein preparations displayed significant pyruvate kinase activity (7 U/mg of protein at the optimum pH 7.5) (Tables 1 and Table 4). We found that the enzyme activity was strongly stimulated (20-fold) in the presence of a set of the RuMP pathway intermediates: glucose-6-phosphate, fructose-6-phosphate, ribose-5-phosphate, ribulose-5-phosphate or erythrose-4-phosphate (Table 4). ATP, PPᵢ and Pᵢ strongly inhibited enzyme activity; however, this inhibitory effect was completely abolished by the addition of activators such as fructose-6-phosphate or ribose-5-phosphate. Overall, the data suggest that the strain possesses an active pyruvate kinase that is strongly dependent on the presence of RuMP cycle intermediates. It should be mentioned that the pyruvate kinase does not show any activity in cell extracts from methanotrophic bacteria, even after supplementation with the inducers shown in Table 1.

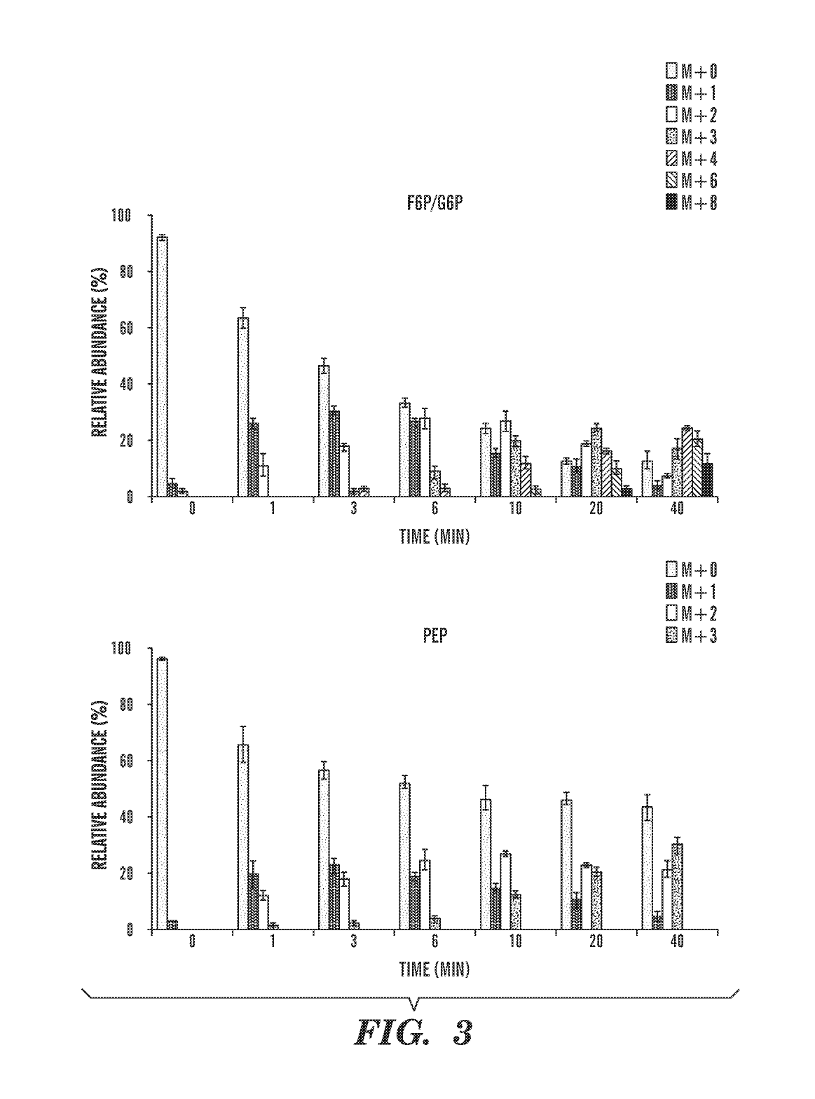

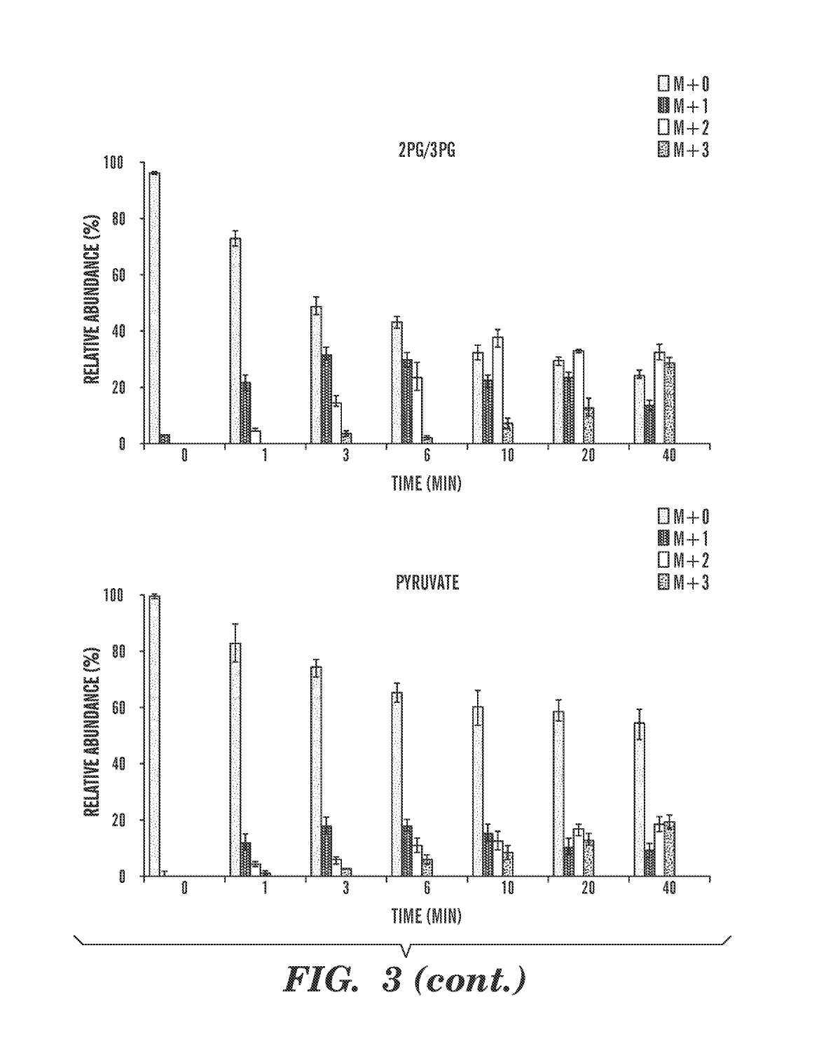

EMP is the main route for C₁-carbon assimilation. In cells of M. alcaliphilum 20Z grown on methane, the intracellular abundance of the majority of EMP pathway intermediates is high (Table 5). In contrast, two key intermediates of the EDD pathway, 6-phosphogluconate and 2-dehydro-3-deoxy-phosphogluconate were only barely detected in cell samples. To further probe the metabolic pathway for methane assimilation in M. alcaliphilum 20Z, we monitored the dynamic incorporation of ¹³C-labeled methane into downstream intermediates of the RuMP pathway (FIG. 3). As expected, the relative abundance of fructose 6-phosphate/glucose 6-phosphate increased at very early time points, demonstrating that methane was rapidly assimilated through the reaction of Ru5P and ¹³C-labeled formaldehyde. The downstream metabolites phosphoglycerate and phosphoenolpyruvate were also sequentially labeled. Due to the low pool size we were not able to estimate the rate of incorporation of ¹³C-carbon into intermediates of the EDD pathway.

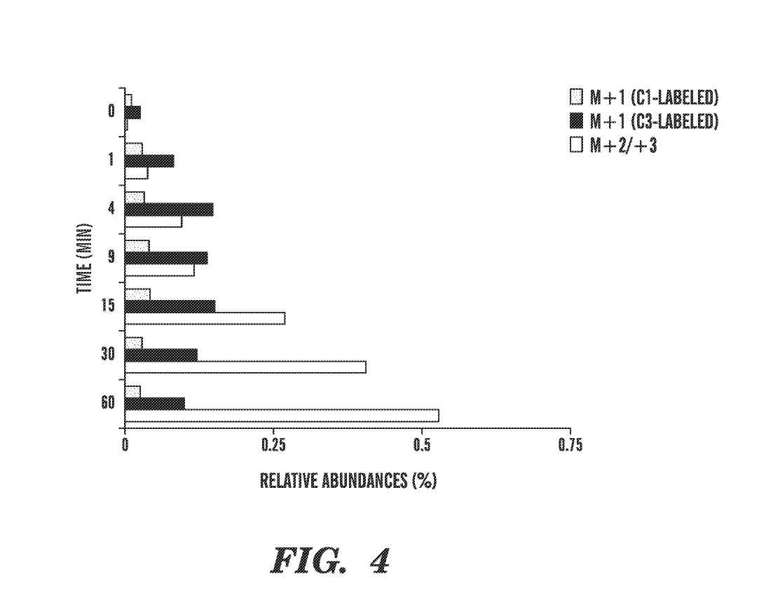

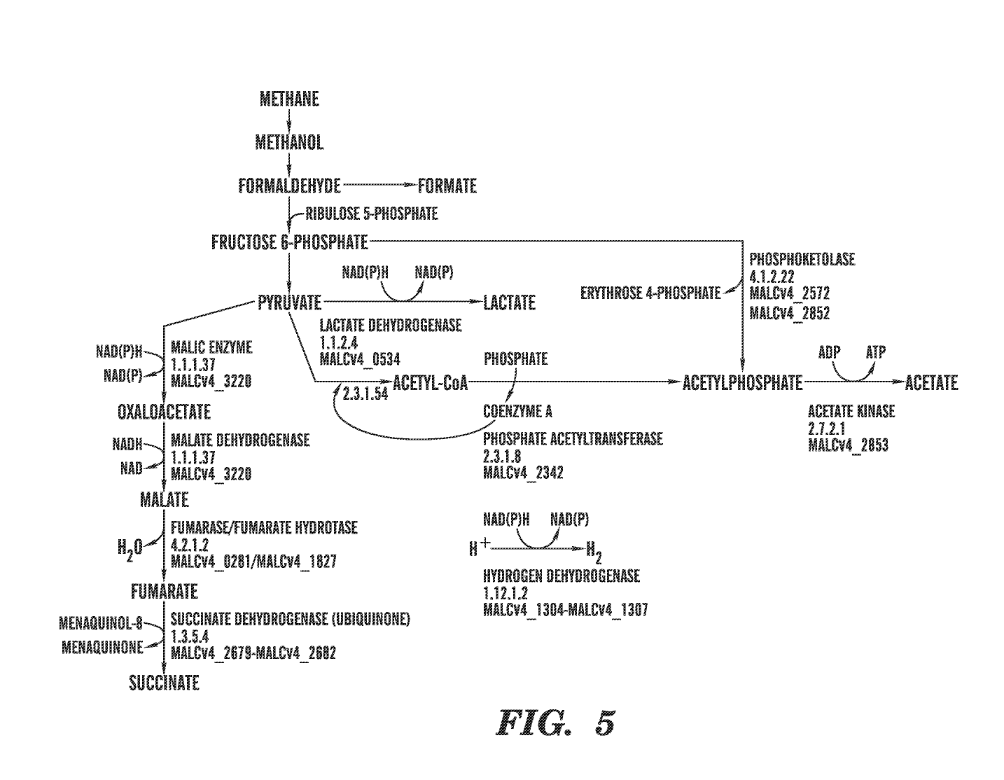

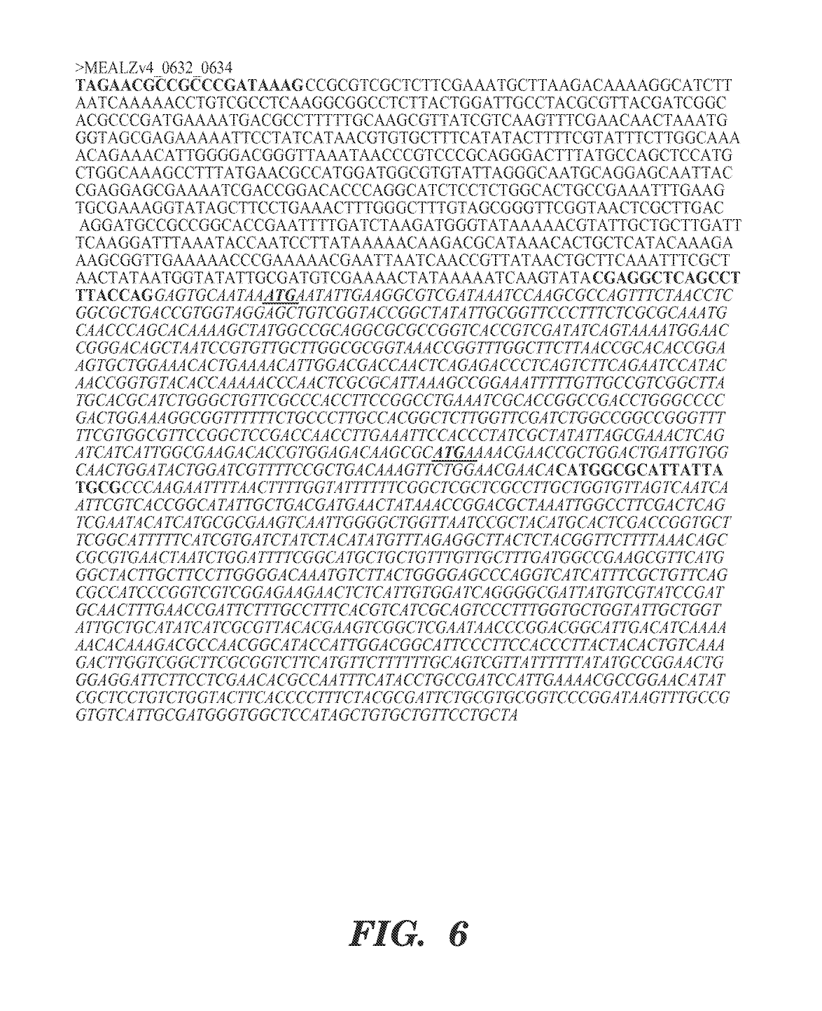

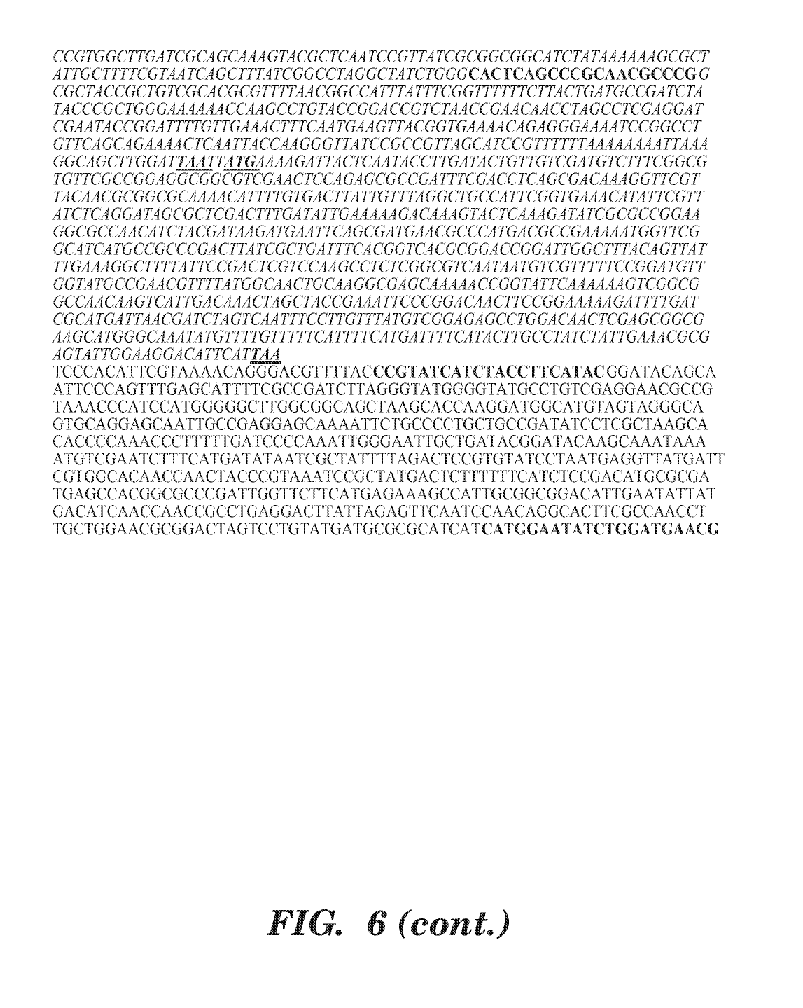

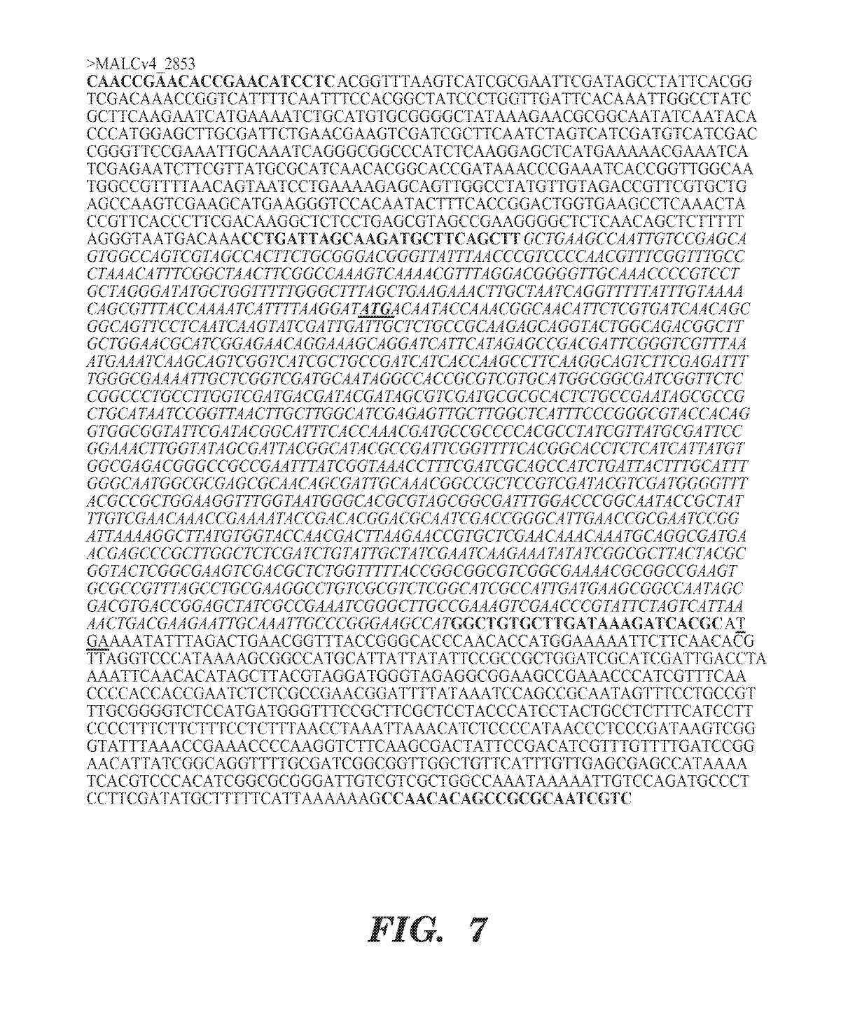

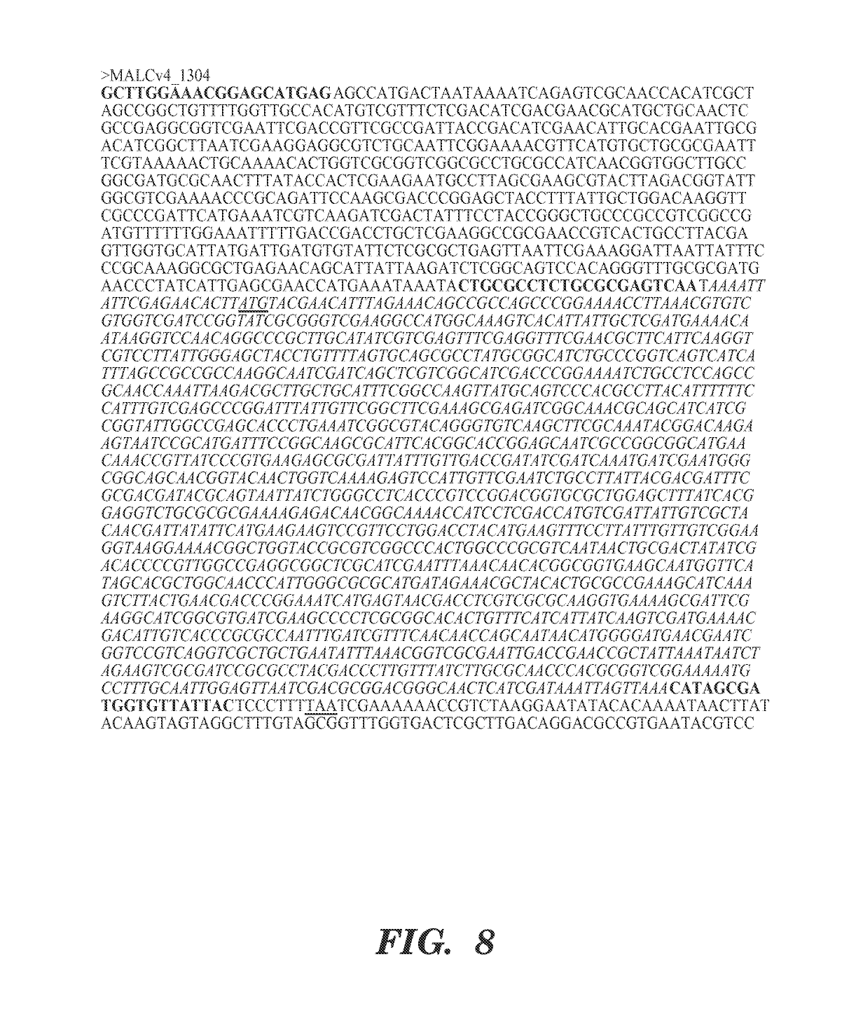

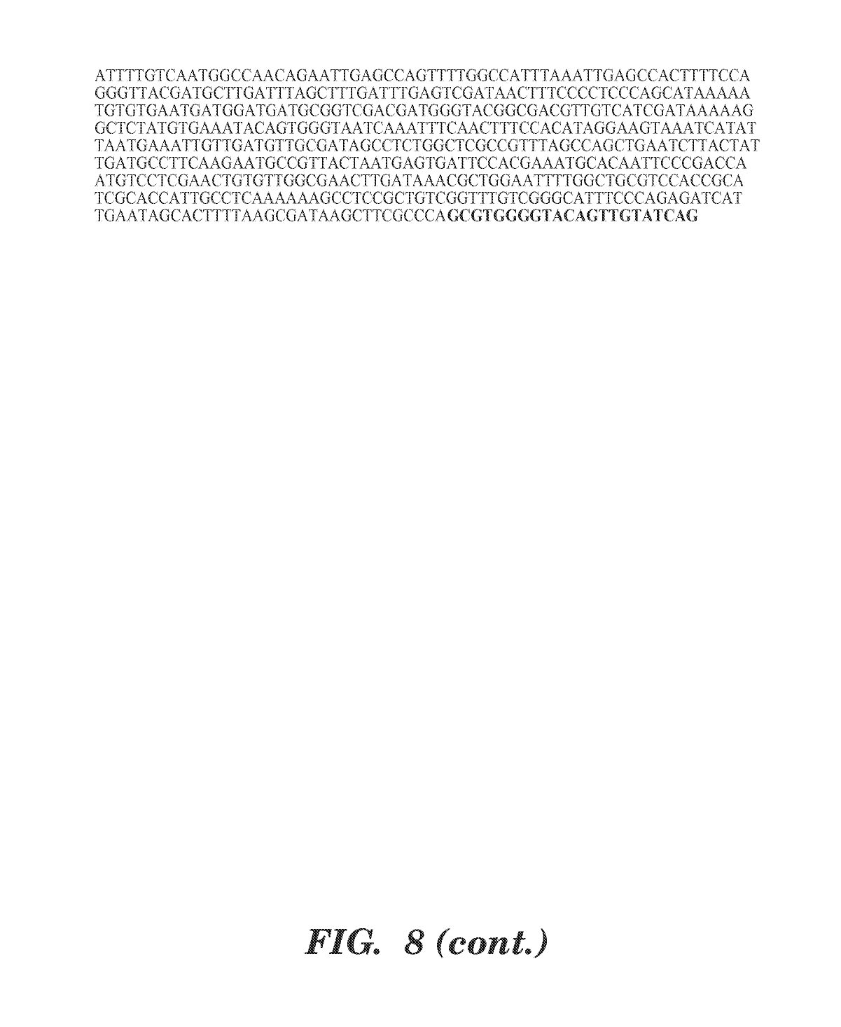

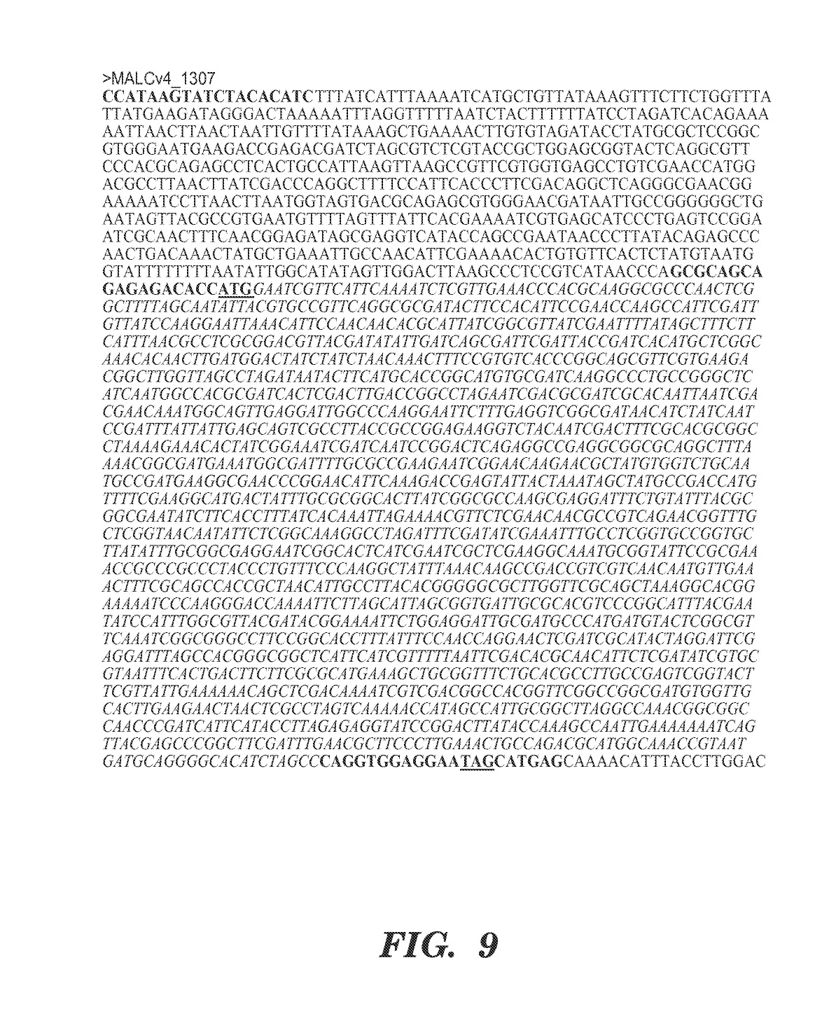

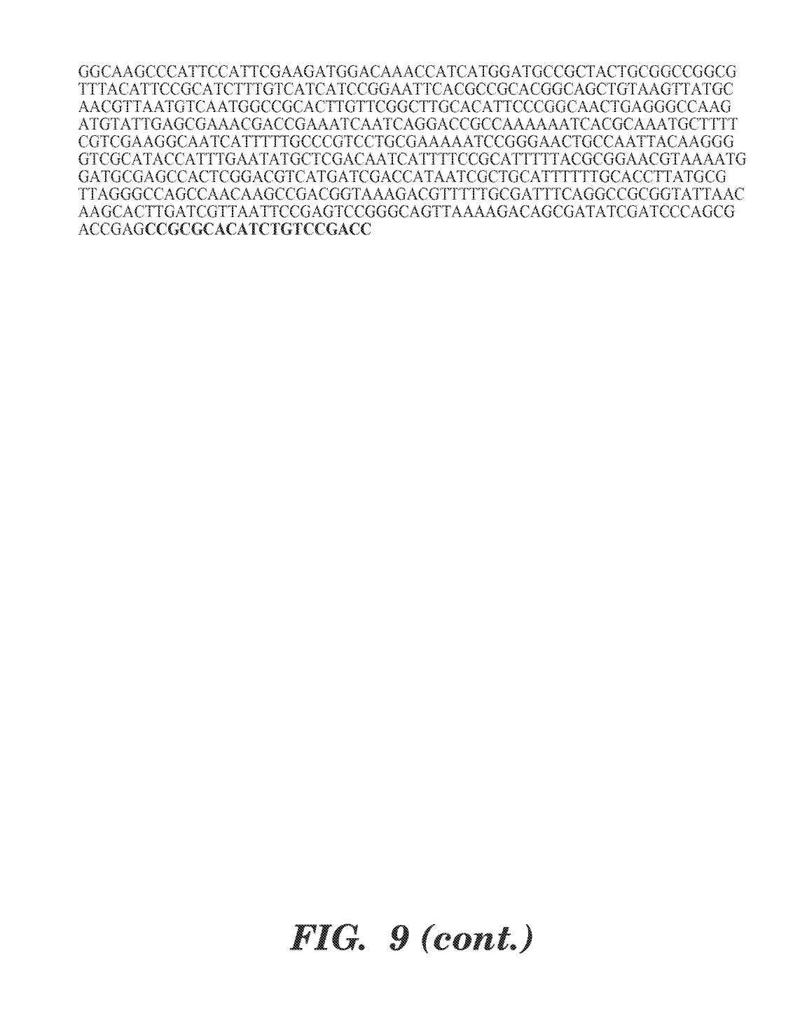

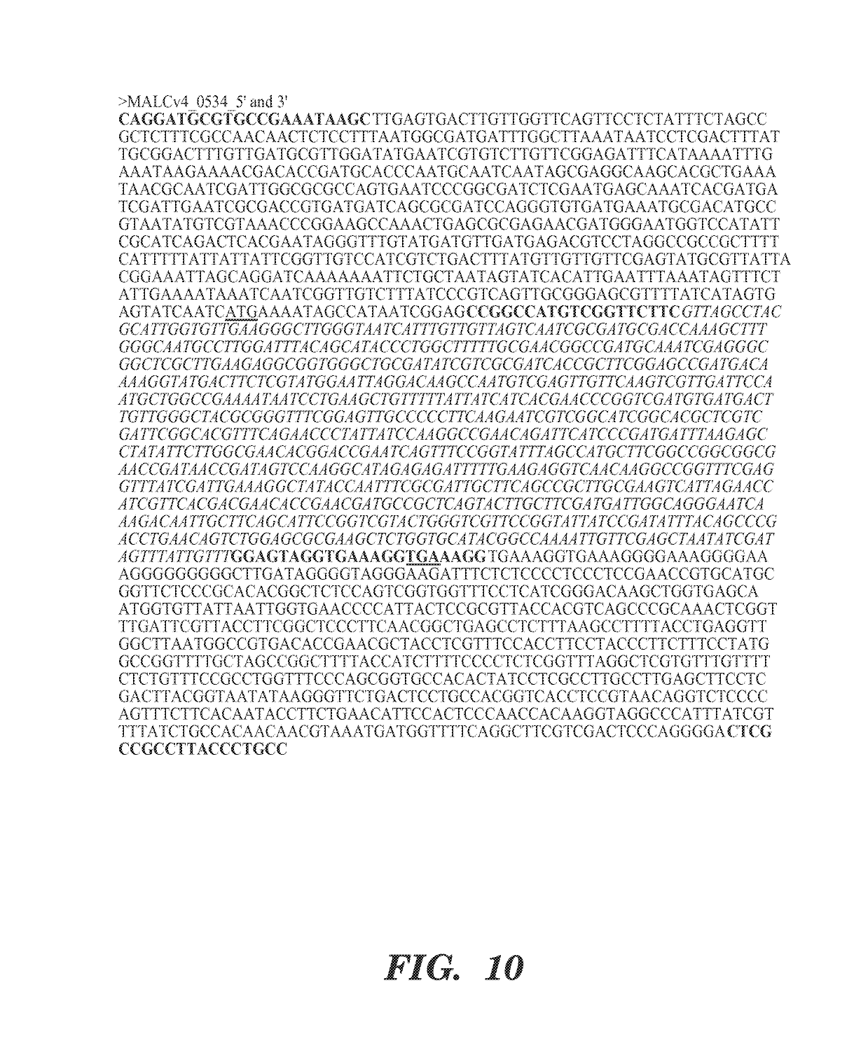

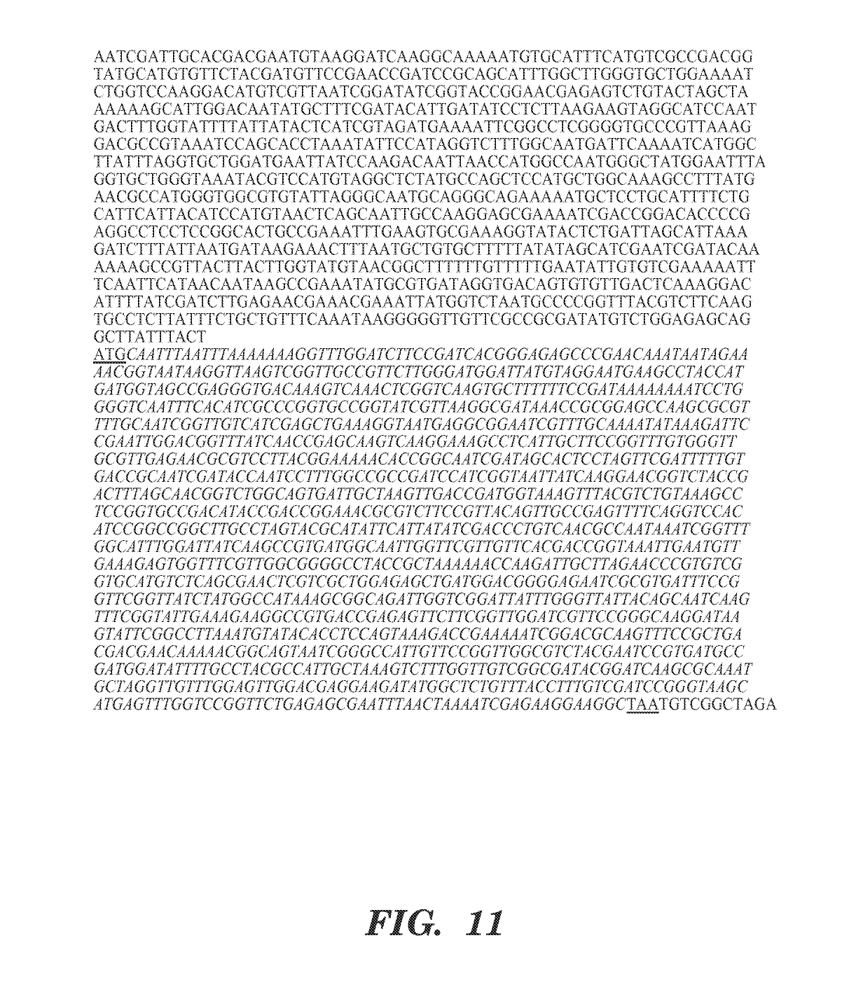

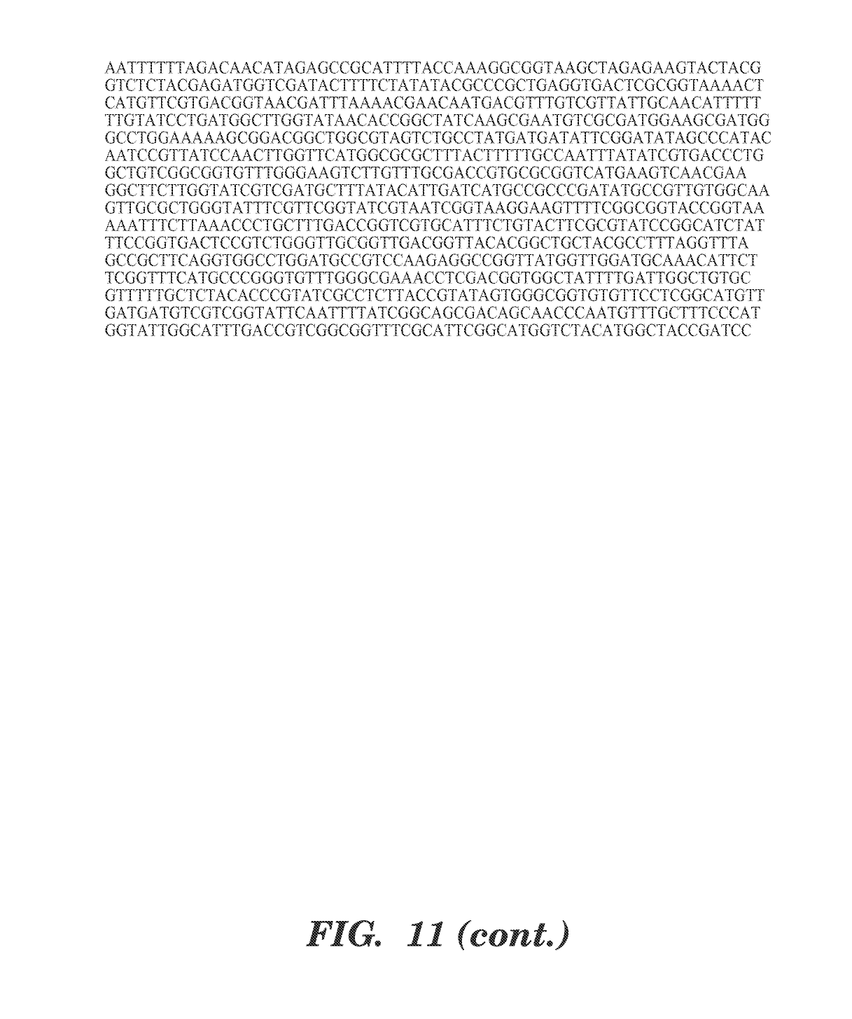

In order to distinguish between the two pathways, we performed ¹³C-pyruvate tracing analysis coupled with tandem mass spectrometry. If pyruvate is formed via the EDD pathway, the initial ¹³C-incorporation should be observed in position 1; in contrast, pyruvate derived from PEP through the EMP pathway should be labeled in position 3 (FIG. 1A). As shown in FIG. 2, only a small fraction of pyruvate was labeled in position 1 during the course of the experiment. The rate of ¹³C incorporation into position 3 of pyruvate was at least six-fold higher than the rate of incorporation into position 1. These experiments confirmed that the major fraction of cellular pyruvate comes from the EMP pathway during growth of the methanotrophic culture on methane. A similar carbon isotopic distribution in pyruvate was observed for Methylomonas sp. LW13, a typical representative of gammaproteobacterial methanotrophic bacteria (FIG. 4).

Methane utilization via fermentation. The new arrangement of the methanotrophic network opens up a possibility for fermentation. Methanotrophs require O₂ for the oxidation of methane, so experiments were carried out with cells grown in bioreactors in which air was provided at low levels and the dissolved O₂ concentrations were kept at undetectable to 0.1%. In a continuous bioreactor culture, M. alcaliphilum 20Z grew slowly, with a doubling time of 23 h. Transcriptomic profiles of batch bioreactor cultures grown at low O₂ showed that similarly to what is observed for aerobic growth, relative expression of EMP-genes is high. The most notable changes in the transcriptome were the down-regulation of genes for NADH:ubiquinone oxidoreductase and cytochrome c oxidase, and the up-regulation of genes for the O₂ carrier bacteriohemerythrin and for the pathways for mixed-acid fermentation and H₂ production (Table 3). In our experiments, the expression profile of bacteriohemerythrin, shown to be essential for high in vitro activity of particulate methane monooxygenase (pMMO) in Methylococcus capsulatus Bath [27], indicated that it most likely contributes to O₂-scavenging/partitioning in M. alcaliphilum 20Z. Genes predicted to encode fermentation pathway enzymes with increased expression under microoxic conditions included a putative acetate kinase, 3-ketoacyl-CoA thiolase, 3-hydroxyacyl-CoA dehydrogenase, malate dehydrogenase, fumarase, succinate dehydrogenase, lactate dehydrogenase and phosphoketolase. Intriguingly, up-regulation of genes encoding a NAD-reducing hydrogenase was observed (Table 3). These changes suggested production of a set of possible fermentation products, including formate, acetate, succinate, lactate, 3-hydroxybutyrate, and H₂. Significantly, in bioreactor cultures acetate, succinate, lactate and H₂ were detected, but only in medium from batch and chemostat cultures grown at low O₂ tension, while formate increased about 3-fold (Table 2). Extracellular concentrations of these acids increased markedly after incubation of low O₂ bioreactor samples in closed vials flushed with N₂. Low amounts of 3-hydroxybutyrate also accumulated. Furthermore, significant accumulation of H₂ was detected in closed vial experiments (Table 2). Similar incubations supplied with ¹³C-methane confirmed that formic and acetic acids were produced from methane (Table 6). The rate of methane consumption in the closed vial experiments was exceptionally low (1.75±0.41 nmol min⁻¹ mg protein⁻¹), however 3 and 15% of the added methane was consumed in 12 and 60 h, respectively. The total amount of produced extracellular carbon, mostly acetate and formate, was equivalent to 40-50% of the total methane carbon consumed. These data indicate that in the presence of sufficient O₂ to drive methane oxidation, M. alcaliphilum 20Z is capable of fermentation from methane-derived formaldehyde, and that methane utilization at low O₂ tension involves switching to a novel fermentation mode leading to the formation of formate, acetate, succinate, lactate, and hydroxybutyrate as end products, with little biomass synthesis (FIG. 3). The presence of putative fermentation genes in the genome of multiple gammaproteobacterial methanotrophs indicates that this type of metabolism is likely widespread (Table 7).

The utilization of the PPi-mediated EMP pathway significantly increases the predicted efficiency of one-carbon assimilation. Genes encoding a membrane-bound proton-translocating pyrophosphatase show significant expression, suggesting that this enzyme could be one of the possible candidates for regeneration of PPi from ATP. The predicted ratio of ATP hydrolysis/PPi formation for this class of enzymes is 1:3 [22]. Therefore, not only does the assimilation of 9 mol of formaldehyde by this metabolic scheme to generate 3 three-carbon intermediates require no additional energy, it actually produces three moles of reducing power (NADH) and two moles of ATP (FIG. 1B).

The discovery of glycolysis-based methane assimilation in strains M. alcaliphilum 20Z and Methylomonas LW13 and production of hydrogen in strain M. alcaliphilum 20Z opens new opportunities to producing a variety of products using methane as a feedstock. Virtually all biosynthetic modules for the production of a wide variety of chemicals developed for glucose-based catalysis in Escherichia coli could also be implemented in cultures containing this EMP variant of the RuMP pathway. Thus, our demonstration of methane-based fermentation provides new approaches for the commercial conversion of methane to hydrogen and excreted products using microoxic production conditions.

Cultivation and growth parameters. M. alcaliphilum 20Z cells were grown using a mineral salts medium [31] in either closed vials (50 ml culture in 250 ml vials, with shaking at 200 rpm) or bioreactor cultures (fed-batch or chemostat; 1 L working volume in a two liter bench top BioFlo™ 110 modular bioreactors, New Brunswick Scientific, Edison, N.J.). Cells were grown at 28-30° C. Optical density of cell cultures was measured on a Beckman DU® 640B spectrophotometer in plastic 1.5 mL cuvettes with a 1 cm path length. Chemostat cultures maintained a steady-state optical density at 600 nm (OD₆₀₀) of approximately 2.0±0.2. The dilution rate was 0.12 h⁻¹ for aerobic cultures (influent gas mixture—20% CH₄: 20% O₂: 60N₂, dissolved O₂ tension was 49-54% or 5% CH₄: 3.5% O₂ balanced with N₂, dissolved O₂ tension was 18-35%) and 0.03 h⁻¹ for low O₂ cultures (influent gas mixture 20% CH₄:5% O₂:75% N₂; dissolved O₂ tension was non-detectable to 0.1%; or 5% CH₄:3.5% O₂ balanced with N₂, dissolved O₂ tension was 0.5-5%). pH (9.0) was controlled by the automatic addition of IN NaOH. Agitation was kept constant at 500-1000 rpm. Samples of inflow and outflow gases were either collected daily in triplicates for gas analysis or were analyzed immediately every 15 minutes using a SRI 8610C™ Gas Chromatograph connected to a bioreactor unit. The rates of methane consumption and H₂ production were determined by incubating cell samples (50 ml, OD₆₀₀ between 2 and 4) in closed 250 ml vials for 12-60 h at 28° C. Prior to incubation vials were flushed for 15 min with a gas mixture containing either 20% CH₄: 5% O₂: 75% N₂ or 20% CH₄: 80% N₂, or 20% ¹³CH₄: 80% N₂.

Gas analysis. Methane measurements were made on a Shimadzu Gas Chromatograph GC-14A, using an FID detector with helium as the carrier gas. CH₄, CO₂, O₂ and H₂ measurements were made on a multiple gas analyzer SRI 8610C™ Gas Chromatograph equipped with TCD/FID detectors (SRI Instruments). Concentrations of gases were deduced from standard curves.

Gene expression experiments. RNA extraction, sequencing, alignment and mapping were performed as described [32].

Metabolite measurement. Metabolic and ¹³C-labeling studies on batch and fed-batch cultures were carried out as described [33] with modification for desalting. Briefly, the dried sample was re-dissolved in 1 mL water and handled according to SPE procedures [34]. MCX, MAX and WAX cartridges (1 cm³, 30 mg, Waters, Milford, Mass., USA) were preconditioned separately. A WAX cartridge was connected beneath a MCX cartridge. Each 1 mL sample was directly loaded through both the MCX and WAX reservoirs. The loaded fraction was collected and made basic with 5% ammonium hydroxide and then loaded into a MAX reservoir, followed by elution. After loading and washing with 1 mL of water, the two adjacent MCX and WAX cartridges were disconnected and eluted separately. All the eluted solutions were dried using a vacuum centrifuge. For LC-MS/MS analysis, each dried sample was re-dissolved in 50 μL water and pooled. LC-MS/MS experiments were carried out on a Waters LC-MS system consisting of a 1525μ binary HPLC pump with a 2777C autosampler coupled to a Quattro Micro API™ triple-quadrupole mass spectrometer (Micromass, Manchester, UK), or a Thermo Scientific TSQ Tquantum access triple stage quadrupole mass spectrometer. The HILIC columns (Luna NH₂, 250 mm×2 mm, 5 μm, and ZIC-HILIC, 150 mm×4.6 mm, 5 μm) employing gradient elution were carried out using the previously described conditions [35-36]. Sugar phosphates were measured by using an ion pairing-reverse phase method [37]. Singly labeled pyruvate position was determined by multiple reaction monitoring (MRM) scan mode with an injection volume of 10 μL. The MRM experiments were carried out as described previously [38]. The dwell time for each MRM transition was 0.08 s. All peaks were integrated using Masslynx Applications Manager™ (version 4.1) software. Quantification of metabolites was obtained by adding culture-derived global ¹³C-labeled internal standards before cell extraction [37]. Relative abundance (%) was obtained by normalizing the pool of each metabolite to the sum of all the targeted metabolites.

Dynamic ¹³C incorporation. For the ¹³C methane tracing experiment, M. alcaliphilus 20Z cells grown to mid-exponential phase (OD600=0.6-0.8) on ¹²C methane in vials or fed-batch bioreactor were rapidly transferred to a fresh flask with the same percentage of ¹³C methane as the sole carbon source as deduced from a growth curve. At the defined time points, the cell culture was harvested and metabolites were analyzed as described above.

Protein purification and characterization. Activities of key enzymes of the central metabolic pathways were measured as described [18, 39-40]. Pyruvate forming activity in cell-free extracts of M. alcaliphilus 20Z was also measured using Pyruvate Assay Kit (BioVision Inc. CA, USA). Recombinant pyruvate kinase PK-ubiqitin-His₆ (“His₆” disclosed as SEQ ID NO: 7) was obtained by cloning of the pyk2 gene (MALCv4_3080) in the vector pHUE and expressing in E. coli BL21 (DE3) cells growing in the presence of 0.5 mM IPTG for 5 hours at 37° C. PK-ubiqitin-His₆ enzyme (“His₆” disclosed as SEQ ID NO: 7) was purified by affinity chromatography on a Ni²⁺-NTA column as described earlier [39].

NMR analysis. To estimate the concentration of metabolites excreted into growth medium, 50 ml samples were collected. Cells were separated by centrifugation (15 min at 2,700×g), filtration via 0.2 μm filter units followed by ultrafiltration through Amicon@Ultra 3K filters. NMR analyses of the culture media were made using a Bruker AVANCE III™ 800 MHz or 700 MHz spectrometer equipped with a cryoprobe or a room temperature probe suitable for ¹H inverse detection with Z-gradients at 298 K. The solvent, water, was removed from the 1 mL culture media samples by drying the samples using a rotary evaporator. The residue was dissolved in an equal volume of phosphate buffer prepared in deuterated water (0.1M; pH=7.4) containing 0.2 mM TSP (3-(trimethylsilyl) propionic-2,2,3,3-d₄ acid sodium salt). From this solution, 600 μL was placed in a 5 mm NMR tube for analysis. One-dimensional ¹H NMR spectra were obtained using a one pulse sequence that included residual water signal suppression from a pre-saturation pulse during the relaxation delay. For each sample, 32 k data points were acquired using a spectral width of 10,000 Hz and a relaxation delay of 6 s. The data were processed using a spectral size of 32 k points and by multiplying with an exponential window function equivalent to a line broadening of 0.3 Hz. The resulting spectra were phase and baseline corrected and referenced with respect to the internal TSP signal. Metabolite peaks in the spectra were then assigned using chemical shift databases, and the peak areas were obtained by integration. Using these peak areas, along with the known concentration of the internal reference (TSP) and the number of protons each peak represented in the molecule, the metabolite concentrations in the culture media were estimated. Similarly, concentrations for the ¹³C labeled bacterial products were estimated using ¹³C satellite peaks of metabolites in the 1H NMR spectra. Bruker Topspin™ version 3.0 and 3.1 software packages were used for NMR data acquisition and processing, respectively.

NMR Analysis.

To estimate the concentration of metabolites excreted into growth medium, 50 ml samples were collected. Cells were separated by centrifugation (15 min at 2,700×g), filtration via 0.2 μm filter units followed by ultrafiltration through Amicon@Ultra 3K filters. NMR analyses of the culture media were submitted to the Northwest Metabolomics Research Center (NW-MRC, depts.washington.edu/nwmrc/Home) for analyses.

Construction of engineered strains are performed as previously described (Ojala et al., 2010). The following cloning vectors are used: pCM 184 [41] and ! or pCM433 [42], pCR2.1 (Invitrogen) or pDrive (Qiagen) for cloning of PCR products. E. coli strains JM 109 (34), S17-1 (Yanish-Perron et al., 1985), Turbo (NEBLabs) and Top 10 (Invitrogen) were routinely cultivated at 37° C. in LuriaBertani (LB) medium (BD). The following antibiotic concentrations were used: Tet, 12.5; Kan 100 mg ml-1; Amp 100 mg ml-1, Rif, 100 mg ml-1. Data from Methylomicrobiumn spp genome projects are used for designing primers flanking upstream and downstream regions of targeted genes. Primers used so far are listed in Table 4. Upstream and downstream fragment are PCR amplified, cloned into pCR2.1 or pDrive, or similar vector, and then subcloned into pCM 184. Each construct are verified by sequencing, Resulted vectors are introduced into a donor strain E. coli S17-1 via standard transformation procedure. The donor strain is grown on LB-agar medium supplemented with appropriate antibiotic and the recipient Methylomicrobium strain grown on NMS-agar medium are mixed in a donor:recipient ratio of 1:1, 1:2, 1:3 or 1:4 and plated on the optimized mating medium. Plates are incubated at 30° C. under methane:air atmosphere for 48 h, and cells are transferred from a mating medium onto selective plates. Rifamycin, high pH and/or 3% salinity are applied for counter-selection against the donor cells. The Kan^{R} recombinants are selected and re-plated onto new plates. The identity of the double-crossover mutants is verified by diagnostic PCR with primers specific to the insertion sites.

### REFERENCES (FOR EXAMPLE 1 AND THE BACKGROUND SECTION)

1. Forster, P. M. & Gregory, J. M. The climate sensitivity and its components diagnosed from Earth radiation budget data. J. Climate, 19, 39-52 (2006).
2. Wuebbler, D. J. & Hayhoe, K. Atmospheric methane and global change. Earth. Sci. Rev. 57, 177-210 (2002).
3. Shindel, D. et al. Simultaneously mitigating near-term climate change and improving human health and food security. Science 335, 183-189 (2012).
4. Jiang, H. et al. Methanotrophs: Multifunctional bacteria with promising applications in environmental bioengineering. Biochem. Eng. J. 49, 277-288 (2010).
5. Wendlandt K. D., Stottmeister U., Helm J., Soltmann B., Jechorek M. & Beck, M. The potential of methane-oxidizing bacteria for applications in environmental biotechnology. Eng. Life. Sci. 10, 87-102 (2010).
6. Olah, G. A., Goeppert, A. & Prakash, G. K. S. Beyond Oil And Gas: The Methanol Economy. Wiley-VCH Verlag Gmb&Co. KGaA, Weiheim (2006).
7. Podkolzin, S. G., Stangland, E. E., Jones, M. E., Peringer, E. & Lercher, J. A. Methyl chloride production from methane over lanthanum-based catalysts. J. Am. Chem. Soc. 129, 2569-76 (2007).
8. Dave, B. C. Prospects for methanol production. In Bioenergy. (J. Wall, C. S. Harwood, & A. L. Demain, Eds). ASM Press, Washington D.C. (2008).
9. Labinger, J. A. Methane activation in homogeneous systems. Fuel Process. Technol. 4, 325-38 (1995).
10. Dalton, H. The Leeuwenhoek Lecture 2000 the natural and unnatural history of methane-oxidizing bacteria. Philos. Trans. R. Soc. Lond. B Biol. Sci. 360, 1207-1222 (2005).
11. Trotsenko, Y. A., Doronina, N. V. & Khmelenina, V. N. Biotechnological potential of aerobic methylotrophic bacteria: a review of current state and future prospects Appl. Biochem. Microbiol. 41, 433-441 (2005).
12. Boden, R. et al. Complete genome sequence of the aerobic marine methanotroph Methylomonas methanica MC09. J. Bacteriol. 193, 7001-7002 (2011).
13. Strom, T., Ferenci, T. & Quayle, J. R. The carbon assimilation pathways of Methylococcus capsulatus, Pseudomonas methanica and Methylosinus trichosporium (OB3b) during growth on methane. Biochem. 144, 465-476 (1974).
14. Anthony, C. The Biochemistry of Methylotrophs. Academic Press, Inc. Ltd., London, United Kingdom (1982).
15. Leak, D. J. & Dalton, H. Growth yields of methanotrophs. Appl. Microbiol. Biot. 23, 477-481 (1986).
16. Trotsenko, Y. A. & Murrell J. C. Metabolic aspects of aerobic obligate methanotrophy. Adv. Appl. Microbiol. 63, 183-229 (2008).
17. Khmelenina, V. N., Kalyuzhnaya, M. G., Starostina, N. G., Suzina, N. E. & Trotsenko, Yu, A. Isolation and characterization of halotolerant alkaliphilic methanotrophic bacteria from Tuva soda lakes. Curr. Microbiol. 35, 257-261 (1997).
18. Rozova, O. N., Khmelenina, V. N., Vuilleumier, S., & Trotsenko, Y. A. Characterization of recombinant pyrophosphate-dependent 6-phosphofructokinase from halotolerant methanotroph Methylomicrobium alcaliphilum 20Z. Res. Microbiol. 161, 861-868 (2010).
19. Harwood, J. H. & Pirt, S. J. Quantitative aspects of growth of the methane oxidizing bacterium Methylococcus capsulatus on methane in shake flask and continuous chemostat culture. J. Appl. Bacteriol. 35, 597-607 (1972).
20. Kao, W. C. et al. Quantitative proteomic analysis of metabolic regulation by copper ions in Methylococcus capsulatus (Bath). J. Biol. Chem. 279, 51554-51560 (2004).
21. Vuilleumier, S., et al. Genome sequence of the haloalkaliphilic methanotrophic bacterium Methylomicrobium alcaliphilum 20Z. J. Bacteriol. 194, 551-552 (2012).
22. Scöcke, L., & Schink, B. Membrane-bound proton-translocating pyrophosphatase of Syntrophus gentianae, a syntrophically benzoate-degrading fermenting bacterium. Eur. J. Biochem. 256, 589-594 (1998).
23. Leak, D. J. & Dalton, H. Growth yields of methanotrophs. I. Effect of copper on the energetics of methane oxidation. Appl. Microbiol. Biot. 23, 470-476 (1986).
24. Morinaga, Y., Yamanaka, S., Yoshimura, M., Takinami, K. & Hirose, Y. Methane metabolism of the obligate methane-utilizing bacterium Methylomonas flagellate, in methane-limited and oxygen-limited chemostat culture. Agric. Biol. Chem. 43, 2452-2458 (1979).
25. Rhee, G. Y. & Fuhs, G. W. Wastewater denitrification with one-carbon compounds as energy source. J. Water Pollut. Control Fed. 50, 2111-2119 (1978).
26. Roslev, P. & King, G. M. Aerobic and anaerobic starvation metabolism in methanotrophic bacteria. Appl. Environ. Microbiol. 61, 1563-1570 (1995).
27. Chen K. H. —C., et al. Bacteriohemerythrin bolsters the activity of the particulate methane monooxygenase (pMMO) in Methylococcus capsulatus (Bath). J. Inorg. Biochem. 111, 10-17 (2012).
28. Auman, A. J., Stolyar, S., Costello, A. M. & Lidstrom, M. E. Molecular characterization of methanotrophic isolates from freshwater lake sediment. Appl. Environ. Microbiol. 66, 5259-5266 (2000).
29. Reim, A., Luke, C., Krause, S., Pratscher, J., & Frenzel, P. One millimetre makes the difference: high-resolution analysis of methane-oxidizing bacteria and their specific activity at the oxic-anoxic interface in a flooded paddy soil. ISME J. 6, 2128-2139 (2012).
30. Ho, A., Vlaeminck, S. E., Ettwig, K. F., Schneider, B., Frenzel, P. & Boon, N. Revisiting methanotrophic communities in sewage treatment plants. Appl. Environ. Microbiol. 79, 2841-2846 (2013).
31. Ojala, D. S., Beck, D. A. C. & Kalyuzhnaya, M. G. Genetic systems for moderately halo(alkali)philic bacteria of the genus Methylomicrobium. Methods Enzymol. 495, 99-118 (2011)
32. Matsen, J. B., Yang, S., Stein, L. Y., Beck, D., & Kalyuzhnaya, M. G. Global molecular analyses of methane metabolism in methanotrophic Alphaproteobacterium, Methylosinus trichosporium OB3b. Part I. Transcriptomic study. Front. Microbiol. 4, 40. doi: 10.3389/finicb.2013.00040 (2013).
33. Yang, S., et al. Global molecular analyses of methane metabolism in methanotrophic Alphaproteobacterium, Methylosinus trichosporium OB3b. Part II. Metabolomics and 13C-labeling study. Front Microbiol. 4, 70. doi: 10.3389/fmicb.2013.00070. (2013).
34. Yang, S., Synovec, R. E., Kalyuzhnaya, M. G., & Lidstrom, M. E. Development of a solid phase extraction protocol coupled with liquid chromatography mass spectrometry to analyze central carbon metabolites in lake sediment microcosms. J. Sep. Sci., 34, 3597-3605 (2011).
35. Yang, S., Sadilek, M., Synovec, R. E., & Lidstrom, M. E. Liquid chromatography-tandem quadrupole mass spectrometry and comprehensive two-dimensional gas chromatography-time-of-flight mass spectrometry measurement of targeted metabolites of Methylobacterium extorquens AM 1 grown on two different carbon sources. J. Chromatogr. A. 1216, 3280-3289 (2009).
36. Schiesel, S., Limmerhofer, M., & Lindner, W. Multitarget quantitative metabolic profiling of hydrophilic metabolites in fermentation broths of β-lactam antibiotics production by HILIC-ESI-MS/MS. Anal. Bioanal. Chem. 396,1655-1679 (2010).
37. Buescher, J. M., Moco, S., Sauer, U., & Zamboni, N. Ultrahigh performance liquid chromatography-tandem mass spectrometry method for fast and robust quantification of anionic and aromatic metabolites. Anal. Chem. 82, 4403-4412 (2010).
38. Yang, S., Sadilek, M., & Lidstrom, M. E. Streamlined pentafluorophenylpropyl column liquid chromatography-tandem quadrupole mass spectrometry and global 13C-labeled internal standards improve performance for quantitative metabolomics in bacteria. J. Chromatogr. A, 1217, 7401-7410 (2010).
39. Reshetnikov, A. S., et al., Characterization of the pyrophosphate-dependent 6-phosphofructokinase from Methylococcus capsulatus Bath. FEMS Microbiol. Lett. 288, 202-10 (2008).
40. Shishkina, V. N., & Trotsenko, Y. A. Multiple enzymatic lesions in obligate methanotrophic bacteria. FEMS Microbiol. Lett. 13, 237-242. (1982).
41. Marx, C. J., Lidstrom, M. E. Broad-host-range cre-lox system for antibiotic marker recycling in gram-negative bacteria. BioTechniques. 2002; 33:1062-1067
42. Marx, C. Development of a broad-host-range sacB-based vector for unmarked allelic exchange. BMC Research Notes 1:1. doi: 10.1186/1756-0500-1-1(2008)

TABLE 1
Kinetic characterisitics of pyruvate kinase 2 from M. alcaliphilum 20Z.

| Substrates | Vmax, U/mg of protein | Kₘ(S_{0.5})*, mM |
|---|---|---|
| PEP in the presence of: | | |
| 2.5 mM ribose-5P | 143.65 ± 4.43 | (1.38 ± 0.06)* |
| 2.5 mM fructose-6P | 179.14 ± 5.59 | (0.12 ± 0,01)* |
| 2.5 mM glucose-6P | 200.1 ± 11.6 | (0.17 ± 0.02)* |
| MgCl₂** | | (1.99 ± 0.17)* |
| ADP** | 135.4 ± 9.45 | 0.16 ± 0.03 |
| UDP** | 82.66 ± 1.97 | 0.16 ± 0.01 |
| CDP** | 132.95 ± 6.02 | 0.39 ± 0.05 |
| GDP** | 99.8 ± 9.5 | 0.43 ± 0.09 |

*S_{0.5} is shown in parentheses and used in place of Kₘ for reactions that do not follow Michaelis-Menten kinetics;
**Measurements were performed in the presence of 2.5 mM ribose-5-phosphate.

TABLE 2
Accumulation of extracellular metabolites (μmol [g DCW⁻¹]).

| | | Micro-aerobic | |
|---|---|---|---|
| | Aerobic bioreactor | bioreactor | Vial |
| Compound | 49-54% dO₂ | (0-0.1% dO₂) | Incubations |
| formate | 687 ± 75 | 1532 ± 272 | 1872 ± 649 |
| | | | (339 ± 184)* |
| acetate | tr | 20.46 ± 0.22 | 504 ± 13 |
| | | | (484 ± 13)* |
| succinate | — | 0.25 ± 0.03 | 6.56 ± 0.78 |
| | | | (6.31 ± 0.7)* |
| lactate | — | 3.8 ± 0.87 | 10.21 ± 1.3 |
| | | | (6.41 ± 1.1)* |
| 3-hydroxybutyrate | — | — | 0.42 ± 0.03 |
| | | | (0.42 ± 0.03)* |
| H₂ | — | 7.9 ± 0.3** | 2237 ± 38 |
| Methane consumed | ND | ND | 2902 ± 68 |

*Numbers in parentheses show increase in the metabolite concentration of micro-aerobic bioreactor culture samples after incubation in a closed vial;
**H₂ concentrations in bioreactor outflow gas (μM);
DCW, dry cell weight;
ND, not done;
dO₂, dissolved O₂;
tr, trace;
—, not detected;

TABLE 3
Gene expression profile in methane-grown cells of M. alcaliphilum 20Z grown in fed-
batch bioreactors.

| | | | | Aerobic | |
|---|---|---|---|---|---|
| | | | | Bioreactor | Fold change |
| | | | | (mean | (Aerobic vs. |
| | Locus tag | Gene | Description | RPKM*) | low O₂) |
| Primary | MALCv4_ | pmoC | particulate methane monooxygenase, C subunit | 61479.46 | 0.98 |
| Oxidation | 0514 | | | | |
| | MALCv4_ | pmoA | particulate methane monooxygenase, A subunit | 32194.25 | 1.41 |
| | 0515 | | | | |
| | MALCv4_ | pmoB | particulate methane monooxygenase, B subunit | 35348.82 | 1.3 |
| | 0516 | | | | |
| | MALCv4_ | mxaI | methanol dehydrogenase, small subunit | 14462.93 | 0.61 |
| | 3445 | | | | |
| | MALCv4_ | mxaF | methanol dehydrogenase, large subunit | 16333.68 | 0.54 |
| | 3448 | | | | |
| | MALCv4_ | fae | formaldehyde-activating enzyme | 8274.29 | 0.21 |
| | 2428 | | | | |
| | MALCv4_ | fdh1A | tungsten-containing NAD-dependent formate | 1300.88 | 0.8 |
| | 1882 | | dehydrogenase, alpha subunit | | |
| | MALCv4_ | nqrE | NADH-quinone reductase subunit E | 1260.08 | 0.31 |
| | 2229 | | | | |
| | MALCv4_ | nqrD | NADH-quinone reductase subunit D | 1375.47 | 0.38 |
| | 2230 | | | | |
| | MALCv4_ | nqrC | NADH-quinone reductase subunit C | 1682.11 | 0.28 |
| | 2231 | | | | |
| | MALCv4_ | nqrB | NADH-quinone reductase subunit B | 1250.3 | 0.34 |
| | 2232 | | | | |
| | MALCv4_ | nqrA | NADH-quinone reductase subunit A | 1440.21 | 0.51 |
| | 2233 | | | | |
| | MALCv4_ | pgi | glucose-6-phosphate isomerase | 263.84 | 0.97 |
| | 0104 | | | | |
| | MALCv4_ | pfp | PPi-dependent phosphofructotransferase | 924 | 0.49 |
| | 3302 | | | | |
| Embden- | MALCv4_ | fbaA | fructose-bisphosphate aldolase | 2479.19 | 0.34 |
| Meyerhof- | 3947 | | | | |
| Parnas | MALCv4_ | tpiA | triosephosphate isomerase | 2493.92 | 0.6 |
| | 1811 | | | | |
| | MALCv4_ | gap | glyceraldehyde 3-phosphate dehydrogenase | 2274.64 | 0.58 |
| | 3079 | | | | |
| | MALCv4_ | pgk | phosphoglycerate kinase | 488.07 | 0.38 |
| | 3549 | | | | |
| | MALCv4_ | pgm3 | 2,3-bisphosphoglycerate-independent | 278.22 | 0.4 |
| | 1633 | | phosphoglycerate mutase | | |
| | MALCv4_ | eno | enolase | 1088.27 | 0.22 |
| | 1462 | | | | |
| | MALCv4_ | pykA | pyruvate kinase II | 1846.02 | 0.42 |
| | 3080 | | | | |
| ED | MALCv4_ | eda | 2-dehydro-3-deoxy-phosphogluconate aldolase | 341.17 | 0.65 |
| D | 1362 | | | | |
| | MALCv4_ | edd | 6-phosphogluconate dehydratase | 225.39 | 0.84 |
| | 1363 | | | | |
| Mixed Acid | MALCv4_ | ldh | lactate dehydrogenase | 48.76 | 1.94 |
| Fermentation | 0534 | | | | |
| | MALCv4_ | xfp | D-fructose 6-phosphate phosphoketolase | 108.35 | 1.6 |
| | 2852 | | | | |
| | MALCv4_ | xfp | D-xylulose 5-phosphate | 55.08 | 1.9 |
| | 2572 | | | | |
| | MALCv4_ | pta | phosphate acetyltransferase | 73.75 | 1.12 |
| | 2342 | | | | |
| | MALCv4_ | ackA | acetate kinase | 88.5 | 0.94 |
| | 2853 | | | | |
| | MALCv4_ | | putative kinase | 260.14 | 15.74 |
| | 0240 | | | | |
| | MALCv4_ | sdhB | succinate dehydrogenase (ubiquinone), Fe—S | 201.42 | 1.46 |
| | 2679 | | protein | | |
| | MALCv4_ | sdhA | succinate dehydrogenase, flavoprotein subunit | 203.59 | 1.78 |
| | 2680 | | | | |
| | MALCv4_ | sdhD | succinate dehydrogenase, hydrophobic | 168.46 | 2.52 |
| | 2681 | | membrane anchor protein | | |
| | MALCv4_ | sdhC | succinate dehydrogenase cytochrome b556 | 127.04 | 3.61 |
| | 2682 | | subunit | | |
| | MALCv4_ | fumC | fumarate hydratase class II (fumarase C) | 63.39 | 5.32 |
| | 0281 | | | | |
| | MALCv4_ | sfcA | NAD-dependent malic enzyme | 87.14 | 1.63 |
| | 1122 | | | | |
| | MALCv4_ | mdh | malate dehydrogenase | 368.53 | 1.63 |
| | 3220 | | | | |
| | MALCv4_ | acnA | aconitate hydratase (aconitase) | 45.04 | 6.98 |
| | 0310 | | | | |
| | MALCv4_ | gltA | citrate synthase 2 | 267.58 | 1.16 |
| | 1360 | | | | |
| | MALCv4_ | gltA | citrate synthase | 221.65 | 0.85 |
| | 3024 | | | | |
| | MALCv4_ | acnB | aconitate hydratase 2 | 357.24 | 0.52 |
| | 3025 | | | | |
| | MALCv4_ | icd | NADP-dependent isocitrate dehydrogenase | 133.36 | 1.41 |
| | 3844 | | | | |
| | MALCv4_ | phbA | 3-hydroxyacyl-CoA dehydrogenase NAD- | 11.61 | 7.04 |
| | 0453 | | binding | | |
| | MALCv4_ | phbB | 3-ketoacyl-CoA thiolase | 15.99 | 4.51 |
| | 0454 | | | | |
| | MALCv4_ | bht | bacteriohemerythrin | 41.5 | 117.06 |
| | 2316 | | | | |
| | MALCv4_ | hoxF | NAD-reducing hydrogenase hoxS subunit alpha | 209.68 | 4.77 |
| | 1307 | | | | |
| H₂- | MALCv4_ | hoxG | NAD-reducing hydrogenase, subunit G, iron- | 284.1 | 3.72 |
| production | 1306 | | sulfur binding | | |
| | MALCv4_ | hoxY | NAD-reducing hydrogenase hoxS subunit delta | 189.08 | 2.73 |
| | 1305 | | | | |
| | MALCv4_ | hoxH | NAD-reducing hydrogenase hoxS subunit beta | 192.8 | 2.51 |
| | 1304 | | | | |

*Values represent reads per kilobase of coding sequence per million (reads) mapped (RPKM).

TABLE 4
Accumulation of extracellular metabolites in bioreactor cultures.

| Effectors | Concentration, mM | Relative activity, %* |
|---|---|---|
| Control (no effector) | — | 100 |
| Ribose-1-phosphate | 5 | 92 |
| Ribose-5-phosphate | 5 | 2092 |
| Ribulose-5-phosphate | 5 | 1890 |
| Glucose-1-phosphate | 5 | 67 |
| Glucose-6-phosphate | 5 | 2545 |
| Fructose-1,6-bisphosphate | 5 | 158 |
| Fructose-6-phosphate | 5 | 2518 |
| Fructose-1-phosphate | 5 | 270 |
| Erythrose-4-phosphate | 5 | 740 |
| Malate | 1 | 82 |
| Citrate | 1 | 140 |
| Serine | 1 | 133 |
| 2-phosphoglycerate | 2 | 75 |
| ATP | 2 | 12 |
| | 1 | 32 |
| | 0.5 | 64 |
| PPi | 2 | 14 |
| Pi | 6 | 11 |
| AMP | 2 | 126 |

*All experiments were performed in triplicates. The standard deviation of the relative activities is ≦5%.

TABLE 5
Intracellular pool of key metabolites in M. alcaliphihum 20Z.

| | Relative |
|---|---|
| Metabolite | abundance (%) |
| Ribulose-5-phosphate/Ribose-5-phosphate | 1.20% ± 0.28% |
| Fructose-1,6-bisphosphate | 1.60% ± 0.42% |
| Fructose-6-phosphate | 3.39% ± 0.31% |
| Glucose-6-phosphate | 2.59% ± 0.18% |
| Glyceraldehyde-3-phosphate/Dihydroxyacetone | 2.60% ± 0.54% |
| 6-Phosphogluconic acid | 0.21% ± 0.04% |
| 2-dehydro-3-deoxy-phosphogluconate | 0.004% ± 0.003% |
| Phosphoglycerate | 5.65% ± 1.12% |
| Phosphoenolpyruvate | 4.28% ± 1.26% |
| Pyruvate | 6.43% ± 1.82% |
| Acetyl-CoA | 0.41% ± 0.12% |
| Succinate | 1.08% ± 0.33% |
| Malate | 2.55% ± 0.62% |
| Fumarate | 0.27% ± 0.07% |
| Citrate | 1.28% ± 0.32% |
| Alanine | 4.08% ± 1.34% |
| Glycerate | 0.64% ± 0.11% |
| Glycine | 7.00% ± 1.40% |
| Serine | 1.40% ± 0.27% |
| Aspartate | 16.04% ± 2.71% |
| Glutamate | 26.37% ± 4.12% |
| Glutamine | 10.93% ± 2.13% |

TABLE 6
Accumulation of extracellular metabolites (mM) from ¹³C-methane in
low O₂ closed vial incubations.

| | | After 12 h |
|---|---|---|
| | Initial | incubation with |
| | concentration | ¹³C-methane |
| ¹²C-Formate | 0.88 ± 0.23 | 0.72 ± 0.07 |
| ¹³C-Formate | 0.01 ± 0.003 | 0.073 ± 0.017 |
| ¹²C-Succinate | 0.00027 ± 0.0002 | 0.00025 ± 0.0001 |
| ¹³C-Succinate | ND | ND |
| ¹²C-Acetate | 0.026 ± 0.001 | 0.036 ± 0.002 |
| ¹³C-Acetate | ND | 0.01 ± 0.001 |
| ¹²C-Lactate | 0.0038 ± 0.0008 | 0.0064 |
| ¹³C-Lactate | ND | ND |

ND, not detected

TABLE 7
Core functional enzymes shared among Type I gammaproteobacterial methanotrophs.

| | Gene | Description | 20Z¹ | 5G² | BG8³ | A45⁴ | AML⁵ | LW14⁶ | IMV⁷ | MC09⁸ | SV96⁹ |
|---|---|---|---|---|---|---|---|---|---|---|---|
| EDD | Eda | 2-dehydro-3-deoxy- | X | X | X | X | X | X | X | X | X |
| Embden- | | phosphogluconate | | | | | | | | | |
| Meyerhof- | | aldolase | | | | | | | | | |
| Parnas | gnd | 6-phosphogluconate | X | X | X | X | X | X | X | X | X |
| | | dehydrogenase, | | | | | | | | | |
| | | decarboxylating | | | | | | | | | |
| | Edd | 6-phosphogluconate | X | X | X | X | X | X | X | X | X |
| | | dehydratase | | | | | | | | | |
| | Pgi | glucose-6-phosphate | X | X | X | X | X | X | X | X | X |
| | | isomerase | | | | | | | | | |
| | Pfp | PPi-dependent | X | X | X | X | X | X | X | X | X |
| | | phosphofructotransferase | | | | | | | | | |
| | | (PPi-dependent | | | | | | | | | |
| | | phosphofructokinase) | | | | | | | | | |
| | Pfk | ATP-dependent | | | | | | | | | X |
| | | phosphofructokinase | | | | | | | | | |
| | fbaA | fructose- | X | X | X | X | X | X | X | X | X |
| | | bisphosphatealdolase | | | | | | | | | |
| | tpiA | triosephosphate | X | X | X | X | X | X | X | X | X |
| | | isomerase | | | | | | | | | |
| | gap | glyceraldehyde 3- | X | X | X | X | X | X | X | X | X |
| | | phosphate | | | | | | | | | |
| | | dehydrogenase | | | | | | | | | |
| | pgk | phosphoglycerate kinase | X | X | X | X | X | X | X | X | X |
| | pgm | phosphoglyceratemutase | X | X | X | X | X | X | X | X | X |
| Mixed Acid | Eno | enolase | X | X | X | X | X | X | X | X | X |
| Fermentation/O₂- | pykA | pyruvate kinase II | X | X | X | X | X | X | X | X | X |
| limitaion/H₂- | Ldh | lactate/malate | X | X | X | | X | X | | | X |
| production | | dehydrogenase | | | | | | | | | |
| | phbA | 3-hydroxyacyl-CoA | X | X | X | X | | X | X | | X |
| | | dehydrogenase NAD- | | | | | | | | | |
| | | binding | | | | | | | | | |
| | phbB | 3-ketoacyl-CoA thiolase | X | X | X | X | X | X | X | | X |
| | Pta | phosphate | X | X | | | | | | X | |
| | | acetyltransferase | | | | | | | | | |
| | ackA | acetate kinase | X | X | X | X | X | X | X | X | X |
| | Xfp | D-fructose 6-phosphate | X | X | | | X | X | | X | X |
| | | phosphoketolase | | | | | | | | | |
| | Xfp | D-xylulose 5- | X | X | X | X | X | X | X | X | X |
| | | phosphate/D-fructose 6- | | | | | | | | | |
| | | phosphate | | | | | | | | | |
| | | phosphoketolase | | | | | | | | | |
| | hoxF | NAD-reducing | X | X | X | X | X | X | X | X | X |
| | | hydrogenase hoxS | | | | | | | | | |
| | | subunit alpha | | | | | | | | | |
| | hoxG | NADH: ubiquinoneoxido | X | X | X | X | X | X | X | X | X |
| | | reductase, subunit G, | | | | | | | | | |
| | | iron-sulfur binding | | | | | | | | | |
| | hoxY | NAD-reducing | X | X | X | X | X | X | X | X | X |
| | | hydrogenase hoxS | | | | | | | | | |
| | | subunit delta | | | | | | | | | |
| | hoxH | NAD-reducing | X | X | X | X | X | X | X | X | X |
| | | hydrogenase hoxS | | | | | | | | | |
| | | subunit beta | | | | | | | | | |
| | sdhB | succinate dehydrogenase | X | X | X | X | X | X | X | X | X |
| | | (ubiquinone), Fe—S | | | | | | | | | |
| | | protein | | | | | | | | | |
| | sdhA | succinate | X | X | X | X | X | X | X | X | X |
| | | dehydrogenase, | | | | | | | | | |
| | | flavoprotein subunit | | | | | | | | | |
| | | succinate | X | X | X | X | X | X | X | X | X |
| | | dehydrogenase, | | | | | | | | | |
| | | hydrophobic membrane | | | | | | | | | |
| | | anchor protein | | | | | | | | | |
| | sdhC | succinate dehydrogenase | X | X | X | X | X | X | X | X | X |
| | | cytochrome b556 | | | | | | | | | |
| | | subunit | | | | | | | | | |
| | fumC | fumarate hydratase class I] | X | X | X | X | X | X | X | | X |
| | | (fumarase C) | | | | | | | | | |
| | sfcA | NAD(P)-dependent | X | X | | X | | X | X | X | |
| | | malic enzyme | | | | | | | | | |
| | mdh | malate dehydrogenase | X | X | X | X | X | X | X | X | X |
| | | bacteriohemerythrin | X | X | X | X | X | X | X | X | X |

¹Methylomicrobium alcaliphilum 20Z;
²Methylomicrobium buryatense 5G;
³Methylomicrobium album BG8;
⁴Methylobacter marinus A45;
⁵Methylosarcina fibrata AML C10;
⁶Methylosarcina lacus LW14;
⁷Methylobacter luteus IMV B 3098T;
⁸Methylomonas methanica MC09;
⁹Methylobacter tundripaludum SV96;
^{10.} O₂ limitation

TABLE 8
Accumulation of extracellular metabolites (μmol L⁻¹ g⁻¹ CDW)

| Description of | | | | | | | | |
|---|---|---|---|---|---|---|---|---|
| modification | Strain | dO₂ | Formate | Acetate | Lactate | Pyruvate | Succinate | Ethanol |
| Original strain, | 20ZER | 0-5% | 4.35 ± 1.05 | 108.4 ± 0.7 | 3.05 ± 0.25 | ND | 0.95 ± 0.25 | ND |
| low O₂ | | | | | | | | |
| Original strain, | 20ZER | 6-40% | TR | ND | ND | ND | ND | ND |
| high O₂ | | | | | | | | |
| Decoupling CH₄ | ΔMtHr | 40% | 14.4 | 31.5 | 61.74 ± 12.43 | 5.1 | 20.5 ± 0.5 | ND |
| oxidation from | | | | | | | | |
| respiration | | | | | | | | |
| Sucrose | Δsps | 5-15% | 34.10 | 364.93 ± 84.37 | 11.53 ± 0.78 | ND | 13.52 ± 2.99 | ND |
| production | | | | | | | | |
| Acetate | Δask | 0-5% | 15.25 ± 2.65 | 1.85 ± 0.35 | 10.45 ± 1.65 | 1.82 ± 0.23 | 3.35 ± 1.25 | 80.2 ± 31 |
| production | | | | | | | | |
| Lactate | Δldh | 0-5% | 6.44 ± 0.01 | 178.73 ± 26.6 | 9.31 ± 3.16 | 0.91 ± 0.33 | 0.75 ± 0.11 | 40.6 |
| production | | | | | | | | |
| Hydrogen | ΔHoxSa | 5-15% | 5.2 ± 0.06 | 66.2 ± 5.87 | 2.5 ± 0.2 | 1.7 ± 0.3 | ND | ND |
| production | | | | | | | | |
| NADH | ΔNDH2 | 5-15% | 17.8 | 15.5 | 5.6 | 0.8 | 0.6 | ND |
| oxidoreductase | | | | | | | | |
| Sucrose | ΔspsΔask | 5-15% | 4.3 ± 0.01 | 15.55 ± 4.65 | 4.85 ± 0.75 | 1.6 ± 0.3 | 4.6 ± 0.01 | 5.8 ± 1.7 |
| production/ | | | | | | | | |
| acetate | | | | | | | | |
| production | | | | | | | | |
| Decoupling/acetate | ΔMtHr | 0-5% | 14.45 ± 2.85 | 146 ± 23 | 4.3 ± 3.0 | ND | 1.65 ± 0.75 | ND |
| production | Δsps | | | | | | | |
| Inhibitor of cyt | 20ZER + | 40% | NT | 3955 ± 230 | 437 ± 30 | NT | NT | NT |
| bc complex | antimycin | | | | | | | |

ND, not detected,
NT, not tested

TABLE 9
Accumulation of extracellular metabolites in aerobic closed
vial incubations. (μmol [g DCW⁻¹]).

| Compound | 20Z | 20ZΔsps | 20ZΔHoxSa | 5GB1 |
|---|---|---|---|---|
| Formate | 896.75 ± | 33.9 ± 5.2 | 12.75 ± 9.0 | 542 ± 133 |
| | 17.75 | | | |
| Acetate | 33.12 ± | 282.19 ± 83.9 | 212.75 ± 62.87 | 54.45 ± 1.03 |
| | 2.94 | | | |
| Succinate | ND | 11.5 ± 0.8 | 10.44 ± 0.94 | 5.7 ± 0.05 |
| Lactate | ND | 13.4 ± 3.0 | ND | ND |

TABLE 10
Accumulation of extracellular metabolites in low O₂ closed
vial incubations. (μmol [g DCW⁻¹]).

| Compound | 20Z | 5GB1 | 5GB1 Δbhr |
|---|---|---|---|
| Formate | 1872 ± 649 | 2933 ± 631 | 3766 ± 447 |
| Acetate | 504 ± 13 | 1113 ± 97 | 1016 ± 308 |
| Succinate | 6.56 ± 0.78 | 147 ± 22 | 143 ± 46 |
| Lactate | 10.21 ± 1.3 | ND | ND |

1. A method for producing at least one excreted product by microbial fermentation of a gaseous substrate, comprising:
a. providing a gaseous substrate comprising CH₄ and optionally, O₂, to a culture of at least one methanotrophic microorganism; and
b. maintaining the microorganism under conditions suitable for fermentation at a dissolved O₂ tension of between 0 and about 1% of saturation with air to produce at least one excreted product; or
maintaining the microorganism under conditions suitable for fermentation at a dissolved O₂ tension of between 0 and about 40% of saturation with air and reducing respiration to produce at least one excreted product.

2. The method of claim 1, wherein the methanotrophic microorganism is a native methanotrophic microorganism.

3. The method of claim 1, wherein reducing respiration comprises contacting the microorganism with an inhibitor of the electron transport chain.

4. The method of claim 3, wherein the inhibitor is antimycin A.

5. A method for producing at least one excreted product by microbial fermentation of a gaseous substrate, comprising:
a. providing a gaseous substrate comprising CH₄ and optionally, O₂, to a culture of at least one methanotrophic microorganism; and
b. maintaining the microorganism under conditions suitable for fermentation to produce at least one excreted product.

6. The method of claim 5, wherein the methanotrophic microorganism is engineered to comprise a downregulated level of a gene selected from the group consisting of:
NAD-reducing hydrogenase (MALCv4_1304 and 1307); acetate kinase (MALCv4_2853); lactate dehydrogenase (MALCv4_0534); acetate kinase (MALCv4_2853) and lactate dehydrogenase (MALCv4_0534); bacteriohemerythrin (MALCv4_2316); sucrose-phosphate synthase (MALCv4_0614); and sucrose-phosphate synthase (MALCv4_0614) and bactenohemerythnn (MALCv4_2316); a member of the cytochrome bc1 complex (MALCv4_0634, MALCv4_0633, and MALCv4_0632); a glycogen biosynthesis gene (MALCv4_3502; MALCv4_3503: MALCv_3504; MALCv4_3505; MALCv_3506; MALCv4_3507, and MALCv_3508); and cytochrome aa3 oxidase (MALCv4_2315).

7. The method of claim 6, wherein the methanotrophic microorganism is engineered to comprise a mutation selected from the group consisting of:
a deletion of NAD-reducing hydrogenase (MALCv4_1304 and 1307); a deletion of acetate kinase (MALCv4_2853); a deletion of lactate dehydrogenase (MALCv4_0534); a deletion of acetate kinase (MALCv4_2853) and lactate dehydrogenase (MALCv4_0534); a deletion of bacteriohemerythrin (MALCv4_2316); and a deletion of sucrose-phosphate synthase (MALCv4_0614); a deletion of sucrose-phosphate synthase (MALCv4_0614) and bacteriohemerythrin (MALCv4_2316); a deletion of a member of the cytochrome bc1 complex (MALCv4_0634, MALCv4_0633, and MALCv4_0632); a deletion of a glycogen biosynthesis gene (MALCv4_3502; MALCv4_3503; MALCv_3504; MALCv4_3505; MALCv_3506; MALCv4_3507, and MALCv_3508); and deletion of cytochrome aa3 oxidase (MALCv4_2315).

8. The method of claim 5, wherein the method further comprises reducing respiration.

9. The method of claim 8, wherein reducing respiration comprises contacting the microorganism with an inhibitor of the electron transport chain.

10. The method of claim 9, wherein the inhibitor is antimycin A.

11. The method of claim 5, wherein the microorganism is maintained under conditions suitable for fermentation at a dissolved O₂ tension of between 0 and about 40% of saturation with air to produce at least one excreted product.

12. The method of claim 5, wherein the dissolved O₂ tension is between 0 and about 10%.

13. The method of claim 12, wherein the dissolved O₂ tension is between 0 and about 1%.

14. The method of claim 13, wherein the dissolved O₂ tension is between 0 and about 0.1%.

15. The method of claim 1, wherein fermentation comprises:
a. converting the gaseous substrate to intracellular formaldehyde; and b. converting the intracellular formaldehyde to at least one excreted product.

16. The method of claim 15, wherein fermentation further comprises reducing respiratory activity.

17. The method of claim 16, wherein respiratory activity is reduced by contacting the cell with a respiratory activity inhibitor or engineering the cell.

18. The method of claim 1, wherein fermentation comprises the conversion of formaldehyde to at least one excreted product by a metabolic pathway in which energy is generated by substrate-level phosphorylation.

19. The method of claim 1, further comprising separating the at least one excreted product from a liquid nutrient media.

20. The method of claim 1, wherein the culture of at least one methanotrophic microorganism is of a genus selected from the group consisting of Methylococcus, Methylomonas, Methylomicrobium, Methylobacter, Methylomarinum, Methylovulum, Methylocaldum, Methylothermus, Methylomarinovum, Methylosphaera, Methylocystis and Methylosinus, and a mixture thereof.

21. The method of claim 1, wherein the culture of at least one methanotrophic microorganism is selected from the group consisting of: Methylomicrobium alcaliphilum; Methylomicrobium buryatense; Methylomonas spp; and a mixture thereof.

22. The method of claim 19, wherein the culture and liquid medium are contained in a bioreactor.

23. The method of claim 19, wherein the culture and liquid medium are contained in a closed vial.

24. The method of claim 1, wherein the ratio of CH₄:O₂ in the gaseous substrate is from about 10:1 to about 1:1

25. The method of claim 24, wherein the ratio of CH₄:O₂ in the gaseous substrate is selected from the group consisting of: about 10:1; about 5:1; about 4:1; about 2:1; about 1.5:1 and about 1:1.

26. The method of claim 1, wherein the at least one excreted product is an organic acid.

27. The method of claim 1, wherein the at least one excreted product is an alcohol.

28. The method of claim 26, wherein the organic acid is selected from the group consisting of: succinate; acetate; butyrate; lactate; malate; fumarate; citrate; glycerate; formic acid; stearic acid; 3-hydroxybutyrate; propionate; and mixtures thereof.

29. The method of claim 27, wherein the alcohol is selected from the group consisting of propanol, isopropanol, ethanol, or mixtures thereof.

30. An engineered methanotrophic bacterium capable of fermenting methane comprising a deletion of one or more of the following genes:
a. NADH-ubiquinone oxidoreductase (MALCv4_1304);
b. hydrogenase (MALCv4_1307);
c. acetate kinase (MALCv4_2853); and
d. lactate dehydrogenase (MALCv4_0534);
e. NAD-reducing hydrogenase (MALCv4_1304 and 1307);
f. bacteriohemelythrin (MALCv4_2316);
g. sucrose-phosphate synthase (MALCv4_0614);
h. a member of the cytochrome bc1 complex (MALCv4_0634, MALCv4_0633, and MALCv4_0632);
i. a glycogen biosynthesis gene (MALCv4_3502; MALCv4_3503; MALCv_3504; MALCv4_3505; MALCv_3506; MALCv4_3507, and MALCv_3508); and
j. cytochrome aa3 oxidase (MALCv4_2315).

31. The engineered methanotrophic bacterium of claim 30, wherein the engineered methanotrophic bacterium is selected from the genus consisting of Methylococcus, Methylomonas, Methylomicrobium, Methylobacter, Methylothermus, Methylocaldum, Methylosphaera, Methylocystis, Methylomarinovum, Methylomicrobium alcaliphilum, Methylomicrobium buryatense and Methylosinus.


SEQUENCE LISTING (8 sequences)

SEQ ID NO: 1 - DNA, 4076 bp, Artificial Sequence
  Description of Artificial Sequence Synthetic polynucleotide
tagaacgccgcccgataaagccgcgtcgctcttcgaaatgcttaagacaaaaggcatcttaatcaaaaacctgtcgcctcaaggcggcctcttactggattgcctacgcgttacgatcggcacgcccgatgaaaatgacgcctttttgcaagcgttatcgtcaagtttcgaacaactaaatgggtagcgagaaaaattcctatcataacgtgtgctttcatatacttttcgtatttcttggcaaaacagaaacattggggacgggttaaataacccgtcccgcagggactttatgccagctccatgctggcaaagcctttatgaacgccatggatggcgtgtattagggcaatgcaggagcaattaccgaggagcgaaaatcgaccggacacccaggcatctcctctggcactgccgaaatttgaagtgcgaaaggtatagcttcctgaaactttgggctttgtagcgggttcggtaactcgcttgacaggatgccgccggcaccgaattttgatctaagatgggtataaaaacgtattgctgcttgatttcaaggatttaaataccaatccttataaaaacaagacgcataaacactgctcatacaaagaaagcggttgaaaaacccgaaaaacgaattaatcaaccgttataactgcttcaaatttcgctaactataatggtatattgcgatgtcgaaaactataaaaatcaagtatacgaggctcagcctttaccaggagtgcaataaatgaatattgaaggcgtcgataaatccaagcgccagtttctaacctcggcgctgaccgtggtaggagctgtcggtaccggctatattgcggttccctttctcgcgcaaatgcaacccagcacaaaagctatggccgcaggcgcgccggtcaccgtcgatatcagtaaaatggaaccgggacagctaatccgtgttgcttggcgcggtaaaccggtttggcttcttaaccgcacaccggaagtgctggaaacactgaaaacattggacgaccaactcagagaccctcagtcttcagaatccatacaaccggtgtacaccaaaaacccaactcgcgcattaaagccggaaatttttgttgccgtcggcttatgcacgcatctgggctgttcgcccaccttccggcctgaaatcgcaccggccgacctgggccccgactggaaaggcggttttttctgcccttgccacggctcttggttcgatctggccggccgggtttttcgtggcgttccggctccgaccaaccttgaaattccaccctatcgctatattagcgaaactcagatcatcattggcgaagacaccgtggagacaagcgcatgaaaacgaaccgctggactgattgtggcaactggatactggatcgttttccgctgacaaagttctggaacgaacacatggcgcattattatgcgcccaagaattttaacttttggtattttttcggctcgctcgccttgctggtgttagtcaatcaattcgtcaccggcatattgctgacgatgaactataaaccggacgctaaattggccttcgactcagtcgaatacatcatgcgcgaagtcaattggggctggttaatccgctacatgcactcgaccggtgcttcggcatttttcatcgtgatctatctacatatgtttagaggcttactctacggttcttttaaacagccgcgtgaactaatctggattttcggcatgctgctgtttgttgctttgatggccgaagcgttcatgggctacttgcttccttggggacaaatgtcttactggggagcccaggtcatcatttcgctgttcagcgccatcccggtcgtcggagaagaactctcattgtggatcaggggcgattatgtcgtatccgatgcaactttgaaccgattctttgcctttcacgtcatcgcagtccctttggtgctggtattgctggtattgctgcatatcatcgcgttacacgaagtcggctcgaataacccggacggcattgacatcaaaaaacacaaagacgccaacggcataccattggacggcattcccttccacccttactacactgtcaaagacttggtcggcttcgcggtcttcatgttcttttttgcagtcgttattttttatatgccggaactgggaggattcttcctcgaacacgccaatttcatacctgccgatccattgaaaacgccggaacatatcgctcctgtctggtacttcacccctttctacgcgattctgcgtgcggtcccggataagtttgccggtgtcattgcgatgggtggctccatagctgtgctgttcctgctaccgtggcttgatcgcagcaaagtacgctcaatccgttatcgcggcggcatctataaaaaagcgctattgcttttcgtaatcagctttatcggcctaggctatctgggcactcagcccgcaacgcccggcgctaccgctgtcgcacgcgttttaacggccatttatttcggttttttcttactgatgccgatctatacccgctgggaaaaaaccaagcctgtaccggaccgtctaaccgaacaacctagcctcgaggatcgaataccggattttgttgaaactttcaatgaagttacggtgaaaacagagggaaaatccggcctgttcagcagaaaactcaattaccaagggttatccgccgttagcatccgttttttaaaaaaaattaaaggcagcttggattaattatgaaaagattactcaataccttgatactgttgtcgatgtctttcggcgtgttcgccggaggcggcgtcgaactccagagcgccgatttcgacctcagcgacaaaggttcgttacaacgcggcgcaaaacattttgtgacttattgtttaggctgccattcggtgaaacatattcgttatctcaggatagcgctcgactttgatattgaaaaagacaaagtactcaaagatatcgcgccggaaggcgccaacatctacgataagatgaattcagcgatgaacgcccatgacgccgaaaaatggttcggcatcatgccgcccgacttatcgctgatttcacggtcacgcggaccggattggctttacagttatttgaaaggcttttattccgactcgtccaagcctctcggcgtcaataatgtcgtttttccggatgttggtatgccgaacgttttatggcaactgcaaggcgagcaaaaaccggtattcaaaaaagtcggcggccaacaagtcattgacaaactagctaccgaaattcccggacaacttccggaaaaagattttgatcgcatgattaacgatctagtcaatttccttgtttatgtcggagagcctggacaactcgagcggcgaagcatgggcaaatatgttttgtttttcattttcatgattttcatacttgcctatctattgaaacgcgagtattggaaggacattcattaatcccacattcgtaaaacagggacgttttacccgtatcatctaccttcatacggatacagcaattcccagtttgagcattttcgccgatcttagggtatggggtatgcctgtcgaggaacgccgtaaacccatccatgggggcttggcggcagctaagcaccaaggatggcatgtagtagggcagtgcaggagcaattgccgaggagcaaaattctgcccctgctgccgatatcctcgctaagcacaccccaaaccctttttgatccccaaattgggaattgctgatacggatacaagcaaataaaatgtcgaatctttcatgatataatcgctattttagactccgtgtatcctaatgaggttatgattcgtggcacaaccaactacccgtaaatccgctatgactcttttttcatctccgacatgcgcgatgagccacggcgcccgattggttcttcatgagaaagccattgcggcggacattgaatattatgacatcaaccaaccgcctgaggacttattagagttcaatccaacaggcacttcgccaaccttgctggaacgcggactagtcctgtatgatgcgcgcatcatcatggaatatctggatgaacg

SEQ ID NO: 2 - DNA, 2662 bp, Artificial Sequence
  Description of Artificial Sequence Synthetic polynucleotide
caaccgaacaccgaacatcctcacggtttaagtcatcgcgaattcgatagcctattcacggtcgacaaaccggtcattttcaatttccacggctatccctggttgattcacaaattggcctatcgcttcaagaatcatgaaaatctgcatgtgcggggctataaagaacgcggcaatatcaatacacccatggagcttgcgattctgaacgaagtcgatcgcttcaatctagtcatcgatgtcatcgaccgggttccgaaattgcaaatcagggcggcccatctcaaggagctcatgaaaaacgaaatcatcgagaatcttcgttatgcgcatcaacacggcaccgataaacccgaaatcaccggttggcaatggccgttttaacagtaatcctgaaaagagcagttggcctatgttgtagaccgttcgtgctgagccaagtcgaagcatgaagggtccacaatactttcaccggactggtgaagcctcaaactaccgttcacccttcgacaaggctctcctgagcgtagccgaaggggctctcaacagctctttttagggtaatgacaaacctgattagcaagatgcttcagcttgctgaagccaattgtccgagcagtggccagtcgtagccacttctgcgggacgggttatttaacccgtccccaacgtttcggtttgccctaaacatttcggctaacttcggccaaagtcaaaacgtttaggacggggttgcaaaccccgtcctgctagggatatgctggtttttgggctttagctgaagaaacttgctaatcaggtttttatttgtaaaacagcgtttaccaaaatcattttaaggatatgacaataccaaacggcaacattctcgtgatcaacagcggcagttcctcaatcaagtatcgattgattgctctgccgcaagagcaggtactggcagacggcttgctggaacgcatcggagaacaggaaagcaggatcattcatagagccgacgattcgggtcgtttaaatgaaatcaagcagtcggtcatcgctgccgatcatcaccaagccttcaaggcagtcttcgagattttgggcgaaaattgctcggtcgatgcaataggccaccgcgtcgtgcatggcggcgatcggttctccggccctgccttggtcgatgacgatacgatagcgtcgatgcgcgcactctgccgaatagcgccgctgcataatccggttaacttgcttggcatcgagagttgcttggctcatttcccgggcgtaccacaggtggcggtattcgatacggcatttcaccaaacgatgccgccccacgcctatcgttatgcgattccggaaacttggtatagcgattacggcatacgccgattcggttttcacggcacctctcatcattatgtggcgagacgggccgccgaatttatcggtaaacctttcgatcgcagccatctgattactttgcatttgggcaatggcgcgagcgcaacagcgattgcaaacggccgctccgtcgatacgtcgatggggtttacgccgctggaaggtttggtaatgggcacgcgtagcggcgatttggacccggcaataccgctatttgtcgaacaaaccgaaaataccgacacggacgcaatcgaccgggcattgaaccgcgaatccggattaaaaggcttatgtggtaccaacgacttaagaaccgtgctcgaacaaacaaatgcaggcgatgaacgagcccgcttggctctcgatctgtattgctatcgaatcaagaaatatatcggcgcttactacgcggtactcggcgaagtcgacgctctggtttttaccggcggcgtcggcgaaaacgcggccgaagtgcgccgtttagcctgcgaaggcctgtcgcgtctcggcatcgccattgatgaagcggccaatagcgacgtgaccggagctatcgccgaaatcgggcttgccgaaagtcgaacccgtattctagtcattaaaactgacgaagaattgcaaattgcccgggaagccatggctgtgcttgataaagatcacgcatgaaaatatttagactgaacggtttaccgggcacccaacaccatggaaaaattcttcaacacgttaggtcccataaaagcggccatgcattattatattccgccgctggatcgcatcgattgacctaaaattcaacacatagcttacgtaggatgggtagaggcggaagccgaaacccatcgtttcaaccccaccaccgaatctctcgccgaacggattttataaatccagccgcaatagtttcctgccgtttgcggggtctccatgatgggtttccgcttcgctcctacccatcctactgcctctttcatccttcccctttcttctttcctctttaacctaaattaaacatctccccataaccctcccgataagtcgggtatttaaaccgaaaccccaaggtcttcaagcgactattccgacatcgtttgttttgatccggaacattatcggcaggttttgcgatcggcggttggctgttcatttgttgagcgagccataaaatcacgtcccacatcggcgcgggattgtcgtcgctggccaaataaaaattgtccagatgccctccttcgatatgctttttcattaaaaaagccaacacagccgcgcaatcgtc

SEQ ID NO: 3 - DNA, 2887 bp, Artificial Sequence
  Description of Artificial Sequence Synthetic polynucleotide
gcttggaaacggagcatgagagccatgactaataaaatcagagtcgcaaccacatcgctagccggctgttttggttgccacatgtcgtttctcgacatcgacgaacgcatgctgcaactcgccgaggcggtcgaattcgaccgttcgccgattaccgacatcgaacattgcacgaattgcgacatcggcttaatcgaaggaggcgtctgcaattcggaaaacgttcatgtgctgcgcgaatttcgtaaaaactgcaaaacactggtcgcggtcggcgcctgcgccatcaacggtggcttgccggcgatgcgcaactttataccactcgaagaatgccttagcgaagcgtacttagacggtattggcgtcgaaaacccgcagattccaagcgacccggagctacctttattgctggacaaggttcgcccgattcatgaaatcgtcaagatcgactatttcctaccgggctgcccgccgtcggccgatgttttttggaaatttttgaccgacctgctcgaaggccgcgaaccgtcactgccttacgagttggtgcattatgattgatgtgtattctcgcgctgagttaattcgaaaggattaattatttcccgcaaaggcgctgagaacagcattattaagatctcggcagtccacagggtttgcgcgatgaaccctatcattgagcgaaccatgaaataaatactgcgcctctgcgcgagtcaataaaattattcgagaacacttatgtacgaacatttagaaacagccgccagcccggaaaaccttaaacgtgtcgtggtcgatccggtatcgcgggtcgaaggccatggcaaagtcacattattgctcgatgaaaacaataaggtccaacaggcccgcttgcatatcgtcgagtttcgaggtttcgaacgcttcattcaaggtcgtccttattgggagctacctgttttagtgcagcgcctatgcggcatctgcccggtcagtcatcatttagccgccgccaaggcaatcgatcagctcgtcggcatcgacccggaaaatctgcctccagccgcaaccaaattaagacgcttgctgcatttcggccaagttatgcagtcccacgccttacattttttccatttgtcgagcccggatttattgttcggcttcgaaagcgagatcggcaaacgcagcatcatcgcggtattggccgagcaccctgaaatcggcgtacagggtgtcaagcttcgcaaatacggacaagaagtaatccgcatgatttccggcaagcgcattcacggcaccggagcaatcgccggcggcatgaacaaaccgttatcccgtgaagagcgcgattatttgttgaccgatatcgatcaaatgatcgaatgggcggcagcaacggtacaactggtcaaaagagtccattgttcgaatctgccttattacgacgatttcgcgacgatacgcagtaattatctgggcctcacccgtccggacggtgcgctggagctttatcacggaggtctgcgcgcgaaaagagacaacggcaaaaccatcctcgaccatgtcgattattgtcgctacaacgattatattcatgaagaagtccgttcctggacctacatgaagtttccttatttgttgtcggaaggtaaggaaaacggctggtaccgcgtcggcccactggcccgcgtcaataactgcgactatatcgacaccccgttggccgaggcggctcgcatcgaatttaaacaacacggcggtgaagcaatggttcatagcacgctggcaacccattgggcgcgcatgatagaaacgctacactgcgccgaaagcatcaaagtcttactgaacgacccggaaatcatgagtaacgacctcgtcgcgcaaggtgaaaagcgattcgaaggcatcggcgtgatcgaagcccctcgcggcacactgtttcatcattatcaagtcgatgaaaacgacattgtcacccgcgccaatttgatcgtttcaacaaccagcaataacatggggatgaacgaatcggtccgtcaggtcgctgctgaatatttaaacggtcgcgaattgaccgaaccgctattaaataatctagaagtcgcgatccgcgcctacgacccttgtttatcttgcgcaacccacgcggtcggaaaaatgcctttgcaattggagttaatcgacgcggacgggcaactcatcgataaattagttaaacatagcgatggtgttattactcccttttaatcgaaaaaaccgtctaaggaatatacacaaaataacttatacaagtagtaggctttgtagcggtttggtgactcgcttgacaggacgccgtgaatacgtccattttgtcaatggccaacagaattgagccagttttggccatttaaattgagccacttttccagggttacgatgcttgatttagctttgatttgagtcgataactttcccctcccagcataaaaatgtgtgaatgatggatgatgcggtcgacgatgggtacggcgacgttgtcatcgataaaaaggctctatgtgaaatacagtgggtaatcaaatttcaactttccacataggaagtaaatcatattaatgaaattgttgatgttgcgatagcctctggctcgccgtttagccagctgaatcttactattgatgccttcaagaatgccgttactaatgagtgattccacgaaatgcacaattcccgaccaatgtcctcgaactgtgttggcgaacttgataaacgctggaattttggctgcgtccaccgcatcgcaccattgcctcaaaaaagcctccgctgtcggtttgtcgggcatttcccagagatcattgaatagcacttttaagcgataagcttcgcccagcgtggggtacagttgtatcag

SEQ ID NO: 4 - DNA, 3070 bp, Artificial Sequence
  Description of Artificial Sequence Synthetic polynucleotide
ccataagtatctacacatctttatcatttaaaatcatgctgttataaagtttcttctggtttattatgaagatagggactaaaaatttaggtttttaatctacttttttatcctagatcacagaaaaattaacttaactaattgttttataaagctgaaaacttgtgtagatacctatgcgctccggcgtgggaatgaagaccgagacgatctagcgtctcgtaccgctggagcggtactcaggcgttcccacgcagagcctcactgccattaagttaagccgttcgtggtgagcctgtcgaaccatggacgccttaacttatcgacccaggcttttccattcacccttcgacaggctcagggcgaacggaaaaatccttaacttaatggtagtgacgcagagcgtgggaacgataattgccggggggctgaatagttacgccgtgaatgttttagtttattcacgaaaatcgtgagcatccctgagtccggaatcgcaactttcaacggagatagcgaggtcataccagccgaataacccttatacagagcccaactgacaaactatgctgaaattgccaacattcgaaaacactgtgttcactctatgtaatggtattttttttaatattggcatatagttggacttaagccctccgtcataacccagcgcagcagagagacaccatggaatcgttcattcaaaatctcgttgaaacccacgcaaggcgcccaactcggcttttagcaatattacgtgccgttcaggcgcgatacttccacattccgaaccaagccattcgattgttatccaaggaattaaacattccaacaacacgcattatcggcgttatcgaattttatagctttcttcatttaacgcctcgcggacgttacgatatattgatcagcgattcgattaccgatcacatgctcggcaaacacaacttgatggactatctatctaacaaactttccgtgtcacccggcagcgttcgtgaagacggcttggttagcctagataatacttcatgcaccggcatgtgcgatcaaggccctgccgggctcatcaatggccacgcgatcactcgacttgaccggcctagaatcgacgcgatcgcacaattaatcgacgaacaaatggcagttgaggattggcccaaggaattctttgaggtcggcgataacatctatcaatccgatttattattgagcagtcgccttaccgccggagaaggtctacaatcgactttcgcacgcggcctaaaagaaacactatcggaaatcgatcaatccggactcagaggccgaggcggcgcaggctttaaaacggcgatgaaatggcgattttgcgccgaagaatcggaacaagaacgctatgtggtctgcaatgccgatgaaggcgaacccggaacattcaaagaccgagtattactaaatagctatgccgaccatgttttcgaaggcatgactatttgcgcggcacttatcggcgccaagcgaggatttctgtatttacgcggcgaatatcttcacctttatcacaaattagaaaacgttctcgaacaacgccgtcagaacggtttgctcggtaacaatattctcggcaaaggcctagatttcgatatcgaaatttgcctcggtgccggtgcttatatttgcggcgaggaatcggcactcatcgaatcgctcgaaggcaaatgcggtattccgcgaaaccgcccgccctaccctgtttcccaaggctatttaaacaagccgaccgtcgtcaacaatgttgaaactttcgcagccaccgctaacattgccttacacgggggcgcttggttcgcagctaaaggcacggaaaaatcccaagggaccaaaattcttagcattagcggtgattgcgcacgtcccggcatttacgaatatccatttggcgttacgatacggaaaattctggaggattgcgatgcccatgatgtactcggcgttcaaatcggcgggccttccggcacctttatttccaaccaggaactcgatcgcatactaggattcgaggatttagccacgggcggctcattcatcgtttttaattcgacacgcaacattctcgatatcgtgcgtaatttcactgacttcttcgcgcatgaaagctgcggtttctgcacgccttgccgagtcggtacttcgttattgaaaaaacagctcgacaaaatcgtcgacggccacggttcggccggcgatgtggttgcacttgaagaactaactcgcctagtcaaaaaccatagccattgcggcttaggccaaacggcggccaacccgatcattcataccttagagaggtatccggacttataccaaagccaattgaaaaaaatcagttacgagcccggcttcgatttgaacgcttcccttgaaactgccagacgcatggcaaaccgtaatgatgcaggggcacatctagcccaggtggaggaatagcatgagcaaaacatttaccttggacggcaagcccattccattcgaagatggacaaaccatcatggatgccgctactgcggccggcgtttacattccgcatctttgtcatcatccggaattcacgccgcacggcagctgtaagttatgcaacgttaatgtcaatggccgcacttgttcggcttgcacattcccggcaactgagggccaagatgtattgagcgaaacgaccgaaatcaatcaggaccgccaaaaaatcacgcaaatgcttttcgtcgaaggcaatcatttttgcccgtcctgcgaaaaatccgggaactgccaattacaaggggtcgcataccatttgaatatgctcgacaatcattttccgcatttttacgcggaacgtaaaatggatgcgagccactcggacgtcatgatcgaccataatcgctgcattttttgcaccttatgcgttagggccagccaacaagccgacggtaaagacgtttttgcgatttcaggccgcggtattaacaagcacttgatcgttaattccgagtccgggcagttaaaagacagcgatatcgatcccagcgaccgagccgcgcacatctgtccgacc

SEQ ID NO: 5 - DNA, 2273 bp, Artificial Sequence
  Description of Artificial Sequence Synthetic polynucleotide
caggatgcgtgccgaaataagcttgagtgacttgttggttcagttcctctatttctagccgctctttcgccaacaactctcctttaatggcgatgatttggcttaaataatcctcgactttattgcggactttgttgatgcgttggatatgaatcgtgtcttgttcggagatttcataaaatttgaaataagaaaacgacaccgatgcacccaatgcaatcaatagcgaggcaagcacgctgaaataacgcaatcgattggcgcgccagtgaatcccggcgatctcgaatgagcaaatcacgatgatcgattgaatcgcgaccgtgatgatcagcgcgatccagggtgtgatgaaatgcgacatgccgtaatatgtcgtaaacccggaagccaaactgagcgcgagaacgatgggaatggtccatattcgcatcagactcacgaatagggtttgtatgatgttgatgagacgtcctaggccgccgcttttcatttttattattattcggttgtccatcgtctgactttatgttgttgttcgagtatgcgttattacggaaattagcaggatcaaaaaaattctgctaatagtatcacattgaatttaaatagtttctattgaaaataaatcaatcggttgtctttatcccgtcagttgcgggagcgttttatcatagtgagtatcaatcatgaaaatagccataatcggagccggccatgtcggttcttcgttagcctacgcattggtgttgaagggcttgggtaatcatttgttgttagtcaatcgcgatgcgaccaaagctttgggcaatgccttggatttacagcataccctggctttttgcgaacggccgatgcaaatcgagggcggctcgcttgaagaggcggtgggctgcgatatcgtcgcgatcaccgcttcggagccgatgacaaaaggtatgacttctcgtatggaattaggacaagccaatgtcgagttgttcaagtcgttgattccaatgctggccgaaaataatcctgaagctgtttttattatcatcacgaacccggtcgatgtgatgacttgttgggctacgcgggtttcggagttgcccccttcaagaatcgtcggcatcggcacgctcgtcgattcggcacgtttcagaaccctattatccaaggccgaacagattcatcccgatgatttaagagcctatattcttggcgaacacggaccgaatcagtttccggtatttagccatgcttcggccggcggcgaaccgataaccgatagtccaaggcatagagagatttttgaagaggtcaacaaggccggtttcgaggtttatcgattgaaaggctataccaatttcgcgattgcttcagccgcttgcgaagtcattagaaccatcgttcacgacgaacaccgaacgatgccgctcagtacttgcttcgatgattggcagggaatcaaagacaattgcttcagcattccggtcgtactgggtcgttccggtattatccgatatttacagcccgacctgaacagtctggagcgcgaagctctggtgcatacggccaaaattgttcgagctaatatcgatagtttattgtttggagtaggtgaaaggtgaaaggtgaaaggtgaaaggggaaaggggaaagggggggggcttgataggggtagggaagatttctctcccctccctccgaaccgtgcatgcggttctcccgcacacggctctccagtcggtggtttcctcatcgggacaagctggtgagcaatggtgttattaattggtgaaccccattactccgcgttaccacgtcagcccgcaaactcggtttgattcgttaccttcggctcccttcaacggctgagcctctttaagccttttacctgaggttggcttaatggccgtgacaccgaacgctacctcgtttccaccttcctacccttctttcctatggccggttttgctagccggcttttaccatcttttcccctctcggtttaggctcgtgtttgttttctctgtttccgcctggtttcccagcggtgccacactatcctcgccttgccttgagcttcctcgacttacggtaatataagggttctgactcctgccacggtcacctccgtaacaggtctccccagtttcttcacaataccttctgaacattccactcccaaccacaaggtaggcccatttatcgttttatctgccacaacaacgtaaatgatggttttcaggcttcgtcgactcccaggggactcgccgccttaccctgcc

SEQ ID NO: 6 - DNA, 3353 bp, Artificial Sequence
  Description of Artificial Sequence Synthetic polynucleotide
aatcgattgcacgacgaatgtaaggatcaaggcaaaaatgtgcatttcatgtcgccgacggtatgcatgtgttctacgatgttccgaaccgatccgcagcatttggcttgggtgctggaaaatctggtccaaggacatgtcgttaatcggatatcggtaccggaacgagagtctgtactagctaaaaaagcattggacaatatgctttcgatacattgatatcctcttaagaagtaggcatccaatgactttggtattttattatactcatcgtagatgaaaattcggcctcggggtgcccgttaaaggacgccgtaaatccagcacctaaatattccataggtctttggcaatgattcaaaatcatggcttatttaggtgctggatgaattatccaagacaattaaccatggccaatgggctatggaatttaggtgctgggtaaatacgtccatgtaggctctatgccagctccatgctggcaaagcctttatgaacgccatgggtggcgtgtattagggcaatgcagggcagaaaaatgctcctgcattttctgcattcattacatccatgtaactcagcaattgccaaggagcgaaaatcgaccggacaccccgaggcctcctccggcactgccgaaatttgaagtgcgaaaggtatactctgattagcattaaagatctttattaatgataagaaactttaatgctgtgctttttatatagcatcgaatcgatacaaaaaagccgttacttacttggtatgtaacggcttttttgtttttgaatattgtgtcgaaaaatttcaattcataacaataagccgaaatatgcgtgataggtgacagtgtgttgactcaaaggacattttatcgatcttgagaacgaaacgaaattatggtctaatgccccggtttacgtcttcaagtgcctcttatttctgctgtttcaaataagggggttgttcgccgcgatatgtctggagagcaggcttatttactatgcaatttaatttaaaaaaaggtttggatcttccgatcacgggagagcccgaacaaataatagaaaacggtaataaggttaagtcggttgccgttcttgggatggattatgtaggaatgaagcctaccatgatggtagccgagggtgacaaagtcaaactcggtcaagtgcttttttccgataaaaaaaatcctggggtcaatttcacatcgcccggtgccggtatcgttaaggcgataaaccgcggagccaagcgcgttttgcaatcggttgtcatcgagctgaaaggtaatgaggcggaatcgtttgcaaaatataaagattccgaattggacggtttatcaaccgagcaagtcaaggaaagcctcattgcttccggtttgtgggttgcgttgagaacgcgtccttacggaaaaacaccggcaatcgatagcactcctagttcgatttttgtgaccgcaatcgataccaatcctttggccgccgatccatcggtaattatcaaggaacggtctaccgactttagcaacggtctggcagtgattgctaagttgaccgatggtaaagtttacgtctgtaaagcctccggtgccgacataccgaccggaaacgcgtcttccgttacagttgccgagttttcaggtccacatccggccggcttgcctagtacgcatattcattatatcgaccctgtcaacgccaataaatcggtttggcatttggattatcaagccgtgatggcaattggttcgttgttcacgaccggtaaattgaatgttgaaagagtggtttcgttggcggggcctaccgctaaaaaaccaagattgcttagaacccgtgtcggtgcatgtctcagcgaactcgtcgctggagagctgatggacggggagaatcgcgtgatttccggttcggttatctatggccataaagcggcagattggtcggattatttgggttattacagcaatcaagtttcggtattgaaagaaggccgtgaccgagagttcttcggttggatcgttccgggcaaggataagtattcggccttaaatgtatacacctccagtaaagaccgaaaaatcggacgcaagtttccgctgacgacgaacaaaaacggcagtaatcgggccattgttccggttggcgtctacgaatccgtgatgccgatggatattttgcctacgccattgctaaagtctttggttgtcggcgatacggatcaagcgcaaatgctaggttgtttggagttggacgaggaagatatggctctgtttacctttgtcgatccgggtaagcatgagtttggtccggttctgagagcgaatttaactaaaatcgagaaggaaggctaatgtcggctagaaattttttagacaacatagagccgcattttaccaaaggcggtaagctagagaagtactacggtctctacgagatggtcgatacttttctatatacgcccgctgaggtgactcgcggtaaaactcatgttcgtgacggtaacgatttaaaacgaacaatgacgtttgtcgttattgcaacatttttttgtatcctgatggcttggtataacaccggctatcaagcgaatgtcgcgatggaagcgatgggcctggaaaaagcggacggctggcgtagtctgcctatgatgatattcggatatagcccatacaatccgttatccaacttggttcatggcgcgctttactttttgccaatttatatcgtgaccctggctgtcggcggtgtttgggaagtcttgtttgcgaccgtgcgcggtcatgaagtcaacgaaggcttcttggtatcgtcgatgctttatacattgatcatgccgcccgatatgccgttgtggcaagttgcgctgggtatttcgttcggtatcgtaatcggtaaggaagttttcggcggtaccggtaaaaatttcttaaaccctgctttgaccggtcgtgcatttctgtacttcgcgtatccggcatctatttccggtgactccgtctgggttgcggttgacggttacacggctgctacgcctttaggtttagccgcttcaggtggcctggatgccgtccaagaggccggttatggttggatgcaaacattcttcggtttcatgcccgggtgtttgggcgaaacctcgacggtggctattttgattggctgtgcgtttttgctctacacccgtatcgcctcttaccgtatagtgggcggtgtgttcctcggcatgttgatgatgtcgtcggtattcaattttatcggcagcgacagcaacccaatgtttgctttcccatggtattggcatttgaccgtcggcggtttcgcattcggcatggtctacatggctaccgatcc

SEQ ID NO: 7 - protein, 6 aa, Artificial Sequence
  Description of Artificial Sequence Synthetic 6xHis tag
HHHHHH

SEQ ID NO: 8 - DNA, 1992 bp, Artificial Sequence
  Description of Artificial Sequence Synthetic polynucleotide
tttcgccgcatttggctggagcagagcatccgccggttttggatgatatatgttcgatttttcaaacgctgcaacaaaatgccgatatcgttttggtcgaaggcgtcggcggttggcgggttccgttgaatgccgactccgatgtctccgatttggcgcgcgtactgggcttgccggtcgtcatcgtggtgaagataaggctgggttgtatcaatcatgcaagattaacttatgagtcgattgtgcagtccgggcaagcttgcgccggttggatagccgtttgctccgataacaacatgctgttgcgcgaggaaaatatcgccacgattaaggcaatgatcgacgcaccgttgctaggagtatttccttttatgcccgaacccgacttcgatgagctgtcggcggcattcgagaaaaccctataaaaaatattgaaattaagttccaaataaaccataattcttttgcggttatttcggcatgcaaaacagtttgcaatgcaaggggagggcgaatgcgccgcattctcataattcaaagccttgacgagtgaaaaggctcaaacaaggcaattaatcaacacaggagaggaaattatggctttaatcacttggacggcagatcaattcggcaccaatgtcggttttgccgacgatgaacataaagttttgttcgacaaactcaataaactttacgatttggcgacaggcggggcggaaagatccgcaatcggcgcgcaactcgatgatttaattgcctatgtcgtagggcatttcgcgcatgaagaaaaagaaatggaagcggccggttttgccggtttggcggcacataaagccgaacacgaggccttagtgggaacttgcgccgatctacaaaaaaaattccatgccggcgaagccgatgtaaccgatgaggtggggcaaatggttaagagttggttggaaagtcacattccgactttcgatagaggctatgccgccgttttgagttaatcgatgaaaaaatgaatggatgctttttcaactttttcaatatgaagttagcatttttcagaaaatatttcgatacggttgtagcctaaattgacggttaaggttactgtcgatgaagactgttagcccggaatagactgtgaaaatgaagataattgaagcccgtgaatagggaatcacgggcttttttgtgcgcgttagttttaaggcatcggcaaagctcttctggcttaccgttgcatcatgggttaaaattctcgtttttaaaatagctattataaaaacagggaaattagtgaatgttgatttgctttgaaccccggctaaaagtagctgatcagcgagagaaactttttctctatttcattgacttaagatagggcgcttgccgttagaaagccaaatcgtacaagccttgatcttaaaacaagctggcgctgatgacgttactaagaatttatcaattgcatacaagagtttctaactaccaccgggaggctgctccgtgaagaaaaccaagcaactgttcgcaagcctgttcttgatgtccttgggactagggaatgcacaggccgaatatacactgaatctgaggcaaggcgtaacccagatcagtcaagaagtctacgatttgcatatgttgattttatggatatgtgtgttaatcggtattgctgtgttcggcacaatgttctactcgatatatcaccatcgtaaatcgaaagggcatgtcgcatctcaatttcatgagagcaccaaagtcgaaattatctggacgatcattccgactttgattctgatcggtatggcgattccggctacaaaaacgatgctcgatttggacgatgttcaagatgcggatatgtcgatcaaagtcaccgggtggcaatggaaatgggaatatgaatacctggataacggtattcactttttcagtaccttggacgaagccagtaatagagcgcgtcagctgggttccggtatcgatccgagatccgtg